(19) United States
(12) Patent Application Publication
(10) Pub. No.: US 2016/0120436 A1
(43) Pub. Date: May 5, 2016

(54) Psychological Evaluation and Methods of Use

(71) Applicant: Neuro-Insight Pty. Ltd., Hawthorn (AU)

(72) Inventor: Richard Bernard SILBERSTEIN, Blackburn (AU)

(73) Assignee: Neuro-Insight Pty. Ltd., Hawthorn (AU)

(21) Appl. No.: 14/993,591

(22) Filed: Jan. 12, 2016

Related U.S. Application Data
(63) Continuation of application No. 14/262,664, filed on Apr. 25, 2014.
Application No. 14/262,664 is a continuation-in-part of application No. 12/520,853, filed on Jun. 22, 2009, now abandoned.
Application No. 14/262,664 is a continuation-in-part of application No. 12/520,857, filed on Jun. 22, 2009, now abandoned.
Application No. 14/262,664 is a continuation-in-part of application No. 12/520,860, filed on Jun. 22, 2009, now abandoned.
Application No. 14/262,664 is a continuation-in-part of application No. 14/080,780, filed on Nov. 14, 2013.
Application No. 14/080,780 is a continuation of application No. 12/520,863, filed on Jun. 22, 2009, now abandoned.
Application No. 14/262,664 is a continuation-in-part of application No. 12/520,868, filed on Jun. 22, 2009, now abandoned.

(51) Int. Cl. A61B 5/0484; A61B 5/00; A61B 5/04

(52) U.S. Cl. CPC A61B 5/04842; A61B 5/04009; A61B 5/04845; A61B 5/4064; A61B 5/04012

(57) ABSTRACT

The psychological impact of entertainment material, visual objects, brands and advertising, commercial communication, the response to an individual presenting a message, or to an individual seeking public office can be assessed using methods employing measurements of SSVEP or SSVER phase increase and gaze tracking of subjects in a variety of ways. Various psychological states may be analyzed in order to accurately predict success and to enable early modification in development stages of products or communication.

This application is a continuation of U.S. application Ser. No. 14/262,664 filed on Apr. 25, 2014 (pending), which is a continuation-in-part of 1) U.S. application Ser. No. 12/520,853 filed on Jun. 22, 2009 (abandoned/revived), which claims priority to PCT Application No. PCT/AU2006/002003 filed on Dec. 22, 2006; 2) U.S. application Ser. No. 12/520,857 filed on Jun. 22, 2009 (abandoned), which claims priority to PCT Application No. PCT/AU2006/002004 filed on Dec. 22, 2006; 3) U.S. application Ser. No. 12/520,860 filed on Jun. 22, 2009 (abandoned), which claims priority to PCT Application No. PCT/AU2006/002005 filed on Dec. 22, 2009; 4) U.S. application Ser. No. 14/080,780 filed on Nov. 14, 2013 (pending), which claims priority to U.S. application Ser. No. 12/520,863 filed on Jun. 22, 2009 (abandoned), which claims priority to PCT Application No. PCT/AU2006/002006 filed on Dec. 22, 2006; and 5) U.S. application Ser. No. 12/520,868 filed on Jun. 22, 2009 (abandoned), which claims priority to PCT Appl. No. PCT/AU2006/002007 filed on Dec. 22, 2006, all of which are incorporated by reference herein in their entirety.

### BACKGROUND

Psychological and/or emotional responses can provide important information for those who develop products and services, the success of which relies on their effects upon the target individuals.

At present, the likely commercial success of newly created entertainment material such as television programs, feature films, and video games, or the response to an individual presenting a message, or to an individual seeking public office, is typically estimated by questionnaires with test audiences or focus groups drawn from test audiences that have viewed the material or the individual. Such methods are now recognized as deficient in tapping the emotional responses of the test audiences. It is emotional responses such as the level of engagement with the material or individual, the sense of excitement, and/or the likeability of various characters, that play a crucial role in the success of the entertainment material.

Computer games and computer based entertainment constitute a large and rapidly growing economic sector. The development costs for the more complex Internet based multiplayer games are a very significant investment for even the largest game development corporations. At present, the likely commercial success of a computer game is determined by asking people to report their impressions of the game and then to make modifications to enhance the playability and enjoyment of the games.

There is a commercial imperative to enhance the effectiveness of various types of visual displays, web sites, and print advertising, as well as enhancing the attractiveness of product design and packaging. At present, eye movement technology is one of the methods used to evaluate individuals' psychological responses to text layout, print advertising, product design, and web site layout. While eye movement technology gives an indication of where gaze or visual attention is directed, it gives no indication of the psychological response associated with the direction of the gaze.

The effectiveness of any piece of commercial communication or advertising depends on whether the appropriate psychological states intended by the advertiser are elicited in the viewer while they perceive the commercial communication, henceforth referred to as advertisement. For example, advertisements require a level of memory encoding at the time that the advertiser's brand appears. Frequently, the advertisement may be designed to elicit particular emotional responses in the target audience. Determining whether advertisements are effective before they are launched is most frequently carried out using a variety of psychological methodologies such as in-depth interviews and focus groups. These are widely considered inadequate in that they rely on verbal responses of the test subjects. Such verbal responses are now considered a poor indicator of emotional states, and the market research industry is considering techniques that rely on brain activity measurements.

The qualities associated in the mind of the consumer with a brand or product are a matter of profound commercial significance. The manner in which a brand or product is perceived by the consumer has a powerful impact on the likely purchase behavior of the consumer. The value attributed to the brand is increasingly an important component of the value of a modern corporation. The value of a brand is fundamentally determined by the attitude of the consumer to that brand. Feelings of trust and loyalty, for example, will be associated with high brand value. Brand managers also seek to associate specific qualities with a brand, for example, car safety with a particular brand of car, or innovation with a certain high-technology manufacturer.

### SUMMARY OF THE INVENTION

The present invention provides a more accurate method of measurement of the likely success of entertainment material, effectiveness of commercial communication, or response to an individual.

Generally speaking, the present invention provides a method that relies on the measurement of brain activity, rather than verbal responses, to determine the psychological and especially the emotional responses to entertainment material, commercial communication, and/or individuals. The present invention utilizes the Steady State Visually Evoked Potential (SSVEP) or Steady State Visually Evoked Response (SSVER) phase increase as a measure of increased brain activity.

According to the present invention there is provided a method for determining the psychological impact of entertainment material having at least first and later episodes, the method including the steps of: (a) presenting a first episode to a target group of subjects; (b) after a predetermined period of time, presenting the later episode to the target group of subjects; (c) determining SSVEP or SSVER phase increase of the target group of subjects whilst the later episode is being presented to the target group of subjects; and (d) evaluating the psychological impact of the entertainment material by reference to the levels of SSVEP or SSVER phase increase determined in step (c).

The invention also provides a method for determining the suitability of an actor from a group of actors for a role in entertainment material including the steps of: (a) causing each of actors to separately perform by reading the same script or acting the same role; (b) presenting each of the actor's performances in step (a) to a test audience; (c) determining SSVEP or SSVER phase increase of the test audience separately for each of the performances; and (d) determining the suitability of the actors for the role by reference to the SSVEP or SSVER phase increase determined in step (c).

The invention also provides a method of determining the selection of a person from a group of persons for a public role, the method including the steps of: (a) causing each person to separately make a presentation which is associated with the public role; (b) presenting the each of the presentations of step (a) to a test audience; (c) determining SSVEP or SSVER phase increase of the test audience separately for each of the persons; and (d) selecting a person for the role by deference to the SSVEP or SSVER phase increase determined in step (c).

The invention also provides a system for determining the psychological impact of entertainment material having at least first and later episodes, the system including: (a) display means for displaying a later episode of the entertainment material to a target group of subjects who have earlier viewed the first episode of the entertainment material; (b) determining means for determining SSVEP or SSVER phase increase of the target group of subjects whilst the later episode is being presented to the target group of subjects; and (c) evaluating means for evaluating the psychological impact of the entertainment material by reference to the levels of SSVEP or SSVER phase increase determined by said determining means.

The invention also provides a method of evaluating actors performing in entertainment material, the method including the steps of: (a) presenting the entertainment material in which one or more actors perform to an audience; (b) determining SSVEP or SSVER phase increase of the audience during presentation of the entertainment material in step (a); (c) averaging SSVEP or SSVER phase increase levels separately for each of the actors when they appear in the entertainment material; and (d) evaluating the psychological impact of each of the actors by reference to the separate SSVEP or SSVER phase increase determined in step (c).

The present invention further provides a technique which enables quantitative evaluation of players' psychological responses to various components of a computer game in order to be able to improve the computer game.

According to the present invention there is provided a method of improving a computer game, the method including the steps of: (a) causing a player to play the computer game in which various game situations are presented to the player during the course of the game; (b) recording game situation parameters corresponding to the various game situations of step (a); (c) determining SSVEP or SSVER phase increase of the player during each of the game situations which are presented to the player; (d) evaluating effectiveness of the game situation parameters by reference to SSVEP or SSVER phase increase determined in step (c) for each of the game situation parameters recorded in step (b); and (e) improving the game by eliminating or modifying those game situations which have low levels of SSVEP or SSVER phase increase as determined in step (d).

The invention also provides a system for assessing entertainment value of a computer game including: (a) a computer upon which the computer game to be assessed can be played, the computer being arranged to record game situation parameters corresponding to various game situations that occur during playing of the computer game; (b) means for determining SSVEP or SSVER phase increase of the player during each of the game situations that occur during playing of the computer game; and (c) means for evaluating the effectiveness of the game situation parameters by reference to SSVEP or SSVER phase increase determined by said means for determining SSVEP or SSVER phase increase for each of the recorded game situation parameters.

The invention provides a method of evaluating the response of a subject to visual features of a visual display, the method including the steps of: (a) presenting a visual display having particular visual features to the subject during a first period; (b) determining SSVEP or SSVER phase increase of the subject during the first period; (c) presenting reference display material to a subject during a second period; (d) determining reference SSVEP or SSVER phase increase of the subject during the second period; (e) tracking the gaze position of at least one of the eyes of the subject on the visual display during the first period; and (f) evaluating the response of the subject to particular visual features of the visual display by determining differences in SSVEP or SSVER phase increase determined between steps (b) and (d) when the gaze of the subject is directed at the particular features.

The invention also provides a system for evaluating the response of a subject to visual features of a visual display, the system including: (a) display means for displaying said visual features to the subject; (b) means for determining SSVEP or SSVER phase increase of the subject at predetermined scalp sites of the subject; (c) gaze tracking means for determining the gaze position of the subject on said display means; (d) detecting means for detecting when the gaze position of the subject impinges on selected visual features; and (e) averaging means for calculating average values of SSVEP or SSVER phase increase for each of the selected visual features when the detecting means detects that the gaze position of the subject impinges on the respective selected visual features.

According to the present invention there is provided a method of quantitatively assessing the effectiveness of an audiovisual, visual or audio advertisement including the steps of: (a) presenting the advertisement to a plurality of subjects, the advertisement having a sequence of audiovisual, visual and/or audio features which occur as a function of time; (b) obtaining, during presentation of the advertisement, EEG signals from the subjects from predetermined scalp sites thereof; (c) calculating SSVEP amplitudes and/or phase differences from EEG signals obtained from said predetermined scalp sites in order to obtain output signals which represent predetermined psychological states of each subject to said features as a function of time; (d) combining the output signals from said subjects to obtain pooled output signals; and (e) displaying the pooled output signals to thereby enable quantitative assessment of the subjects' responses to said features of the advertisement in order to assess the effectiveness of the features of the advertisement.

The invention also provides a system for quantitatively assessing the effectiveness of an audiovisual, visual or audio advertisement including: (a) display means for presenting the advertisement to a plurality of subjects, the advertisement having a sequence of audiovisual, visual or audio features which occur as a function of time; (b) means for obtaining, during presentation of the advertisement, EEG signals from said at least one subject from predetermined scalp sites of said subjects; and (c) means for calculating SSVEP amplitudes and/or phase differences from signals obtained from the predetermined sites in order to obtain output signals which represent said predetermined psychological states of said at least one subject to said features as a function of time, to thereby enable quantitative assessment of said subjects' responses to said features of the advertisement in order to assess the effectiveness of the features of the advertisement.

The invention also provides a method of measuring steady-state visually evoked potential (SSVEP) or steady-state visual evoked response (SSVER) of a subject including the step of applying time varying flicker signals only to the peripheral vision regions of the retina of a subject and not applying said time varying flicker signals to the center of vision (fovea) of the subject.

According to the invention there is provided a method of evaluating characteristics of a brand or product including the steps of: (a) presenting the brand or product to the subject during a first period; (b) determining SSVEP or SSVER phase increase of the subject during the first period; (c) presenting neutral visual and/or audio material to a subject during a second period; (d) determining a reference level of SSVEP or SSVER phase increase of the subject during the second period; and (e) evaluating attributes associated by the subject with the brand or product by determining differences in SSVEP or SSVER phase increase between said first and second periods.

According to another aspect of the invention there is provided a method of determining attributes associated with a brand or product including the steps of: (a) simultaneously presenting the brand or product and a semantic probe to the subject during a first period; (b) determining SSVEP or SSVER phase increase of the subject during the first period; (c) presenting the neutral visual and/or audio material to a subject during a second period; (d) determining a reference level of SSVEP or SSVER phase increase of the subject during said second period; and (e) determining whether there is congruence or incongruence between the attributes associated with the brand or product and the semantic probe by assessing whether there is an increase or decrease in SSVEP or SSVER phase increase in step (b) compared to step (d).

The invention also provides a system for determining attributes associated with a brand or product including: (a) display means for displaying the brand or product image to a subject; (b) SSVEP or SSVER phase increase determining means for determining SSVEP or SSVER phase increase of the subject; and (c) assessment means coupled to receive first output signals from said SSVEP or SSVER phase increase determining means in a first period in which the brand or product image is displayed to the subject and to receive second output originals from said SSVEP or SSVER phase increase determining means in a second period in which neutral material is displayed to the subject in order to establish a reference level of SSVEP or SSVER phase increase, the assessment means being operable to assess differences between said first and second output signals.

The invention also provides a system for determining attributes associated with a brand or product including: (a) display means for displaying the brand or product image to a subject; (b) SSVEP or SSVER phase increase determining means for determining SSVEP or SSVER phase increase of the subject; and (c) assessment means coupled to receive first output signals from said SSVEP or SSVER phase increase determining means in a first period in which the brand or product image is displayed to the subject simultaneously with a semantic probe and to receive second output originals from said SSVEP or SSVER phase increase determining means in a second period in which neutral material is displayed to the subject in order to establish a reference level of SSVEP or SSVER phase increase, the assessment means being operable to assess differences between said first and second output signals.

### BRIEF DESCRIPTION OF THE DRAWINGS

FIG. 1 is a schematic view of a system of the invention.

FIG. 2 is a schematic view showing in more detail the manner in which visual flicker stimuli are presented to a subject.

FIG. 3 is a graph showing the opacity of the screen as a function of radius.

FIG. 4 graphically shows the measures for viewing engagement for male and female subjects for different types of entertainment material.

FIG. 5 graphically shows different measures of impact for three different actors.

FIG. 6 shows correlation between the techniques of the invention and known assessment techniques.

FIG. 7 is a schematic diagram of a system of the invention.

FIG. 8 is a schematic view showing in more detail the manner in which visual flicker stimuli are presented to a subject including the location of the infra-red diode and infra-red transistor.

FIG. 9 is a schematic view of the operation of the TrackIR head tracking system.

FIG. 10 is a schematic view of the locations of the infra-red diode and infra-red transistor for the infra-red oculography system.

FIG. 11 is a flowchart illustrating a typical way in which features of a visual display are evaluated, in accordance with the method of the invention.

FIG. 12 shows an example of a visual object in the form of as perfume bottle.

FIGS. 13 and 14 are graphs showing levels of psychological responses to parts of a visual object.

FIG. 15 is a schematic view illustrating the International 10-20 System of electrode locations.

FIG. 16 is a diagrammatic representation showing opacity as a function of radius of a screen which is used in the system of the invention.

FIG. 17 represents still frames from a video advertisement.

FIGS. 18 to 23 illustrate data obtained from subjects as a function of time.

FIG. 24 diagrammatically shows the timing of the presentation of a brand or product image and a semantic probe.

FIG. 25 is a graphical representation of SSVEP or SSVER phase increase as a function of time.

FIG. 26 is a graph showing peak values of SSVEP or SSVER phase increase for three different brands.

FIG. 27 is a graphical representation illustrating brand congruence associated with three different brands.

### DETAILED DESCRIPTION

### Measuring SSVEP or SSVER Phase Increase

### Brain Activity

A number of methods are available for measuring brain activity. The main feature they must possess is adequate temporal resolution or the capacity to track the rapid changes in brain activity. Spontaneous brain electrical activity or the electroencephalogram (EEG) or the brain electrical activity evoked by a continuous visual flicker that is the Steady State Visually Evoked Potential (SSVEP) are two examples of brain electrical activity that can be used to measure changes in brain activity with sufficient temporal resolution. The equivalent spontaneous magnetic brain activity or the magnetoencephalogram (MEG) and the brain magnetic activity evoked by a continuous visual flicker Steady State Visually Evoked Response (SSVER).

### Electroencephalogram and Magnetoencephalogram (EEG and MEG)

The EEG and MEG are the record of spontaneous brain electrical and magnetic activity recorded at or near the scalp surface. Brain activity can be assessed from the following EEG or MEG components.

### 1. Gamma or High Frequency EEG or MEG Activity

This is generally defined as EEG or MEG activity comprising frequencies between 35 Hz and 80 Hz. Increased levels of Gamma activity are associated with increased levels of brain activity, especially concerned with perception. (Fitzgibbon, S. P. et al., “Cognitive tasks augment gamma EEG power,” Clin Neurophysiol., 115(2004):1802-1809).

Where scalp EEG gamma activity is used as the indicator of brain activity, the relevant scalp recording sites are indicated herein. If EEG gamma activity at the specific brain regions listed herein is used as the indicator brain activity, then inverse mapping techniques such as LORETA must be used (Pascual-Marqui, R. D. et al., “Low resolution electromagnetic tomography: a new method for localizing electrical activity in the brain,” Int J Psychophysiol 18(1994):49-65).

If MEG gamma activity at the specific brain regions listed herein is used as the indicator of brain activity, then a multi-detector MEG recording system must be used in conjunction with an MEG inverse mapping technique (see Uutela, K. et al., “Visualization of magnetoencephalographic data using minimum current estimate,” Neuroimage 10(1999):173-180 and Fuchs, M. et al., “Linear and nonlinear current density reconstructions,” J Clin Neurophysiol 16(1999):267-295).

### 2. Frequency of EEG or MEG Alpha Activity

Brain activity may also be indexed by changes in the frequency of the ongoing EEG or MEG in the alpha frequency range (e.g., 8.0 Hz-13.0 Hz). Increased instantaneous frequency in the alpha frequency range is an indication of increased brain activity. The frequency needs to be estimated with high temporal resolution. Two techniques that can be used to measure ‘instantaneous frequency’ are complex demodulation (Walter, D., “The Method of Complex Demodulation,” Electroencephalog. Clin. Neurophysiol, 1968 Suppl 27:53-7) and the use of the Hilbert Transform (Cohen, L., “Time frequency analysis,” pages 27-31, Prentice-Hall, 1995).

Where the frequency of scalp EEG alpha activity is used as the indicator of brain activity, the relevant scalp recording sites are listed herein. If the frequency of EEG alpha activity at the specific brain regions listed herein is used as the indicator brain activity, then inverse mapping techniques such as LORETA must be used (Pascual-Marqui, R. D. et al., “Low resolution electromagnetic tomography: a new method for localizing electrical activity in the brain,” Int J Psychophysiol 18(1994):49-65).

If the frequency of MEG alpha activity at the specific brain regions listed herein is used as the indicator of brain activity, then a multi-detector MEG recording system must be used in conjunction with an MEG inverse mapping technique (see Uutela, K. et al., “Visualization of magnetoencephalographic data using minimum current estimate,” Neuroimage 10(1999):173-180 and Fuchs, M. et al., “Linear and nonlinear current density reconstructions,” J Clin Neurophysiol 16(1999):267-295).

### 3. SSVEP or SSVER Phase as an Indicator of Brain Activity

Brain activity may also be indicated by the phase of the Steady State Visually Evoked Potential (SSVEP) or the Steady State Visually Evoked Response (SSVER).

U.S. Pat. Nos. 4,955,388; 5,331,969; and 6,792,304 (the contents of which are incorporated herein by reference) disclose techniques for obtaining a steady state visually evoked potential (SSVEP) from a subject. This technique can also be used to obtain a steady state visually evoked response (SSVER). These patents disclose the use of Fourier analysis in order to rapidly obtain the SSVEP and SSVER phase and changes thereto. The preferred ways in which SSVEP and SSVER amplitudes and phases are calculated are summarized below.

### SSVEP and SSVER Amplitude and Phase

The digitized brain electrical activity (electroencephalogram or EEG) or brain magnetic activity (MEG) together with timing of the stimulus zero crossings enables one to calculate the SSVEP or SSVER elicited by the flicker at a particular stimulus frequency from the recorded EEG or MEG or from EEG or MEG data that has been pre-processed using Independent Components Analysis (ICA) to remove artifacts and increase the signal to noise ratio. (Bell, A. J. et al., “An information maximisation approach to blind separation and blind deconvolution,” Neural Computation, 7, 6(1995): 1129-1159; Jung, T. et al., “Independent component analysis of single-trial event-related potential,” Human Brain Mapping, 14(2001):168-85).

Calculation of SSVEP or SSVER amplitude and phase coefficients for each stimulus cycle for a given stimulus frequency can be accomplished using Fourier techniques using Equations 1 and 2 below.

$${a}_{n} = \frac{1}{S Δ τ} {\sum }_{i = 0}^{S - 1} f ( nT + i Δ τ ) cos ( \frac{2 π}{T} ( nT + i Δ τ ) ) \\ {b}_{n} = \frac{1}{S Δ τ} {\sum }_{i = 0}^{S - 1} f ( nT + i Δ τ ) sin ( \frac{2 π}{T} ( nT + i Δ τ ) ) \qquad Equation 1$$

Calculation of SSVEP or SSVER Fourier components where aₙ and bₙ are the cosine and sine Fourier coefficients respectively, n represents the nth stimulus cycle, S is the number of samples per stimulus cycle (typically 16 samples per cycle), Δτ is the time interval between samples, T is the period of one cycle and f(nT+iΔτ) is the EEG or MEG signal (raw or pre-processed using ICA).

$${SSVEP}_{amplitude} = \sqrt{( {A}_{n}^{2} + {B}_{n}^{2} )} or \\ {SSVER}_{amplitude} = \sqrt{( {A}_{n}^{2} + {B}_{n}^{2} )} \\ {SSVEP}_{phase} = atan ( \frac{{B}_{n}}{{A}_{n}} ) or {SSVER}_{phase} = atan ( \frac{{B}_{n}}{{A}_{n}} ) \qquad Equation 2$$

where Aₙ and Bₙ are overlapping smoothed Fourier coefficients calculated by using Equation 3 below, where N is the number of Fourier coefficients averaged.

$${A}_{n} = {\sum }_{i = 1}^{i = N} {a}_{n + i} / N \\ {B}_{n} = {\sum }_{i = 1}^{i = N} {b}_{n + i} / N \qquad Equation 3$$

Amplitude and phase components can be calculated using either single cycle Fourier coefficients (aₙ and bₙ) or coefficients that have been calculated by smoothing across multiple cycles (Aₙ and Bₐ).

Equations 2 and 3 describe the procedure for calculating the smoothed SSVEP or SSVER coefficients for a single subject. For pooled data, the SSVEP or SSVER coefficients (Aₙ and Bₙ) for a given electrode are averaged (or pooled) across all of the subjects or a selected group of subjects.

As the number of cycles used in the smoothing increases, the signal to noise ratio increases while the temporal resolution decreases. The number of cycles used in the smoothing is typically in excess of 5 and less than 130.

Equations 2 and 3 apply to scalp SSVEP and SSVER data as well as brain electrical activity inferred at the cortical surface adjacent to the skull and deeper regions. Activity in deeper regions of the brain such as the orbito-frontal cortex or ventro-medial cortex can be determined using a number of available inverse mapping techniques such as brain electrical source analysis (BESA) (BESA GmbH, Freihamer Str. 18, 82166 Grafelfing, Germany), electromagnetic source estimation (EMSE) (Source Signal Imaging, Inc, 2323 Broadway, Suite 102, San Diego, Calif. 92102, USA) and low resolution electromagnetic tomography (LORETA) (Pascual-Marqui, R. D. et al., “Low resolution electromagnetic tomography: a new method for localizing electrical activity in the brain,” Int J Psychophysiol 18(1994):49-65).

### Pre-Determined Psychological States and SSVEP or SSVER Phase Increase

### 1. Engagement

Engagement is determined by the weighted average of SSVEP or SSVER phase increase in four frontal and prefrontal sites. This is given by the following expression:

Engagement=(b₁*SSVEP or SSVER phase increase at electrode F₃+b₂*SSVEP or SSVER phase increase at electrode Fₚ₁+b₃*SSVEP or SSVER phase increase at electrode F₄+b₄*SSVEP or SSVER phase increase at electrode Fₚ₂) Equation 4
where b₁=0.1, b₂=0.4, b₃=0.1, b₄=0.4

If inverse mapping techniques are used, the relevant expression is:

Engagement=(d₁*SSVEP or SSVER phase increase at the left orbito frontal cortex (in vicinity of Brodmann area 11)+d₂*SSVEP or SSVER phase increase at the left dorso-lateral prefrontal cortex (in vicinity of Brodmann area 9)+d₃*SSVEP or SSVER phase increase at the right orbito frontal cortex (in vicinity of Brodmann area 11)+d₄*SSVEP or SSVER phase increase at the right dorso-lateral prefrontal cortex (in vicinity of Brodmann area 9)) Equation 5
where d₁=0.1, d₂=0.4, d₃=0.1, d₄=0.4

### 2. Attraction—Repulsion

Attraction-Repulsion (sometimes termed like-dislike) is given by the difference between SSVEP or SSVER phase increase at left frontal/prefrontal and right frontal/prefrontal regions. Attraction is indicated by larger SSVEP or SSVER phase increase in the left hemisphere compared to the right, while Repulsion is indicated by larger SSVEP or SSVER phase increase in the right hemisphere compared to the left, as given by the following expression:

Attraction=(a₁*SSVEP or SSVER phase increase at electrode F₃+a₂*SSVEP or SSVER phase increase at electrode Fₚ₁−a₃*SSVEP or SSVER phase increase at electrode F₄−a₄*SSVEP or SSVER phase increase at electrode Fₚ₂) Equation 6
where a₁=a₂=a₃=a₄=1.0

If inverse mapping techniques are used, the relevant expression is:

Attraction=(c₁*SSVEP or SSVER phase increase at left orbito-frontal cortex (in vicinity of Brodmann area 11)+c₂*SSVEP or SSVER phase increase at left dorso-lateral prefrontal cortex (in vicinity of Brodmann area 9)−c₃*SSVEP or SSVER phase increase at right orbito-frontal cortex (in vicinity of Brodmann area 11)−c₄*SSVEP or SSVER phase increase at right dorso-lateral prefrontal cortex (in vicinity of Brodmann area 9)) Equation 7
where c₁=1, c₂=1, c₃=1, c₄=1

### 3. Visual Attention to Detail

SSVEP phase increase at the left occipital region, preferably electrode O₁, has been found to be relevant to the assessment of the level of the subject's Visual Attention to Detail. If inverse mapping techniques are used, the relevant location in the left cerebral cortex is the vicinity of Brodmann area 17. Examples of Visual Attention to Detail include text and numbers, as well as details of a scene or face, for example.

### 4. Visual Attention to Global Features

SSVEP phase increase at the right occipital region, preferably electrode O₂, has been found to be relevant to the assessment of the level of the subject's Visual Attention to Global Features. If inverse mapping techniques are used, the relevant location in the right cerebral cortex is the vicinity of Brodmann area 17. Examples of Visual Attention to Global Features include responses to facial expressions, for example.

### 5. Multi-Modal Attention to Detail or Desirability

SSVEP phase increase at the left parietal region, preferably at electrode P₃, has been found to be relevant to the assessment of the level of the subject's Multi-Modal Attention to Detail (Desire) for the subject matter. If inverse mapping techniques are used, the relevant location in the left cerebral cortex is the vicinity of the left intraparietal area. This measure refers to multi-modal or multi-sensory attention. Typically this includes both auditory and visual attention to detail in the visual domain or speech in the auditory domain. When this attention measure is also associated with objects, it indexes the level of desirability associated with said object.

### 6. Multi-Modal Attention to Global Features or Desirability

SSVEP phase increase at the right parietal region, preferably at electrode P₄, has been found to be relevant to the assessment of the level of the subject's Multi-Modal Attention to Global Features (Desire) for the subject matter. If inverse mapping techniques are used, the relevant location in the right cerebral cortex is the vicinity of the right intraparietal area. This measure refers to multi-modal or multi-sensory attention. Typically this includes both auditory and visual attention to global features such as facial expression, scenery in the visual domain, and music in the auditory domain. When this attention measure is also associated with objects, it indexes the level of desirability associated with said object.

### 7. Emotional Intensity

SSVEP phase increase at the right parieto-temporal region, preferably from a single electrode which is approximately equidistant from right hemisphere electrodes O₂, P₄, and T₆, has been found to be relevant to the assessment of the level of the subject's Emotional Intensity response. If inverse mapping techniques are used, the relevant location in the right cerebral cortex is the vicinity of the right parieto-temporal junction. This measure indicates the intensity of the emotional state experienced by the subject. This measure is independent of the specific emotion such as joy, fear, anger, anxiety, etc.

### 8. Long Term Memory Encoding for Details and Verbal Memories/Behavioral Intent

SSVEP phase increase at the left frontal region, preferably approximately equidistant from left hemisphere electrodes C₃, F₃ and F₇ has been found to be relevant to the assessment of the level of the subject's Long Term Memory Encoding for Details response. If inverse mapping techniques are used, the relevant location in the left cerebral cortex is the vicinity of Brodmann areas 6, 44, 45, 46 and 47.

### 9. Long Term Memory Encoding for Emotional and Non-Verbal Memories/Behavioral Intent

SSVEP phase increase at the right frontal region, preferably approximately equidistant from left hemisphere electrodes C₄, F₄ and F₈ has been found to be relevant to the assessment of the level of the subject's Long Term Memory Encoding for Emotional and Non-verbal Memories. If inverse mapping techniques are used, the relevant location in the right cerebral cortex is the vicinity of Brodmann areas 6, 44, 45, 46 and 47.

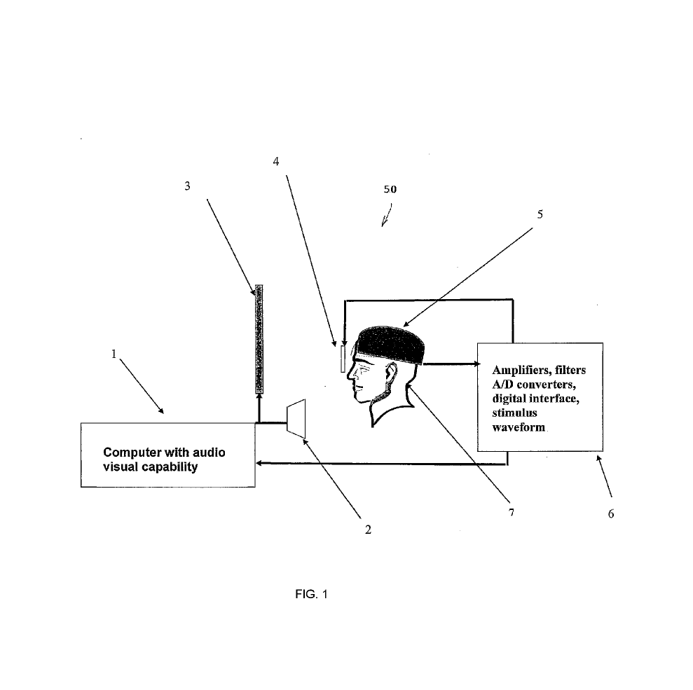

FIG. 1 schematically illustrates a system 50 for determining the response of a subject or a group of subjects to audio-visual material presented on a video screen 3 and loudspeaker 2. The system includes a computer 1 which controls various parts of the hardware and also performs computation on signals derived from the SSVEP or SSVER phase increase of the subject 7, as will be described below. The computer 1 also holds the images and sounds which can be presented to one or more subjects 7 on the screen 3 and/or through the loudspeaker 2.

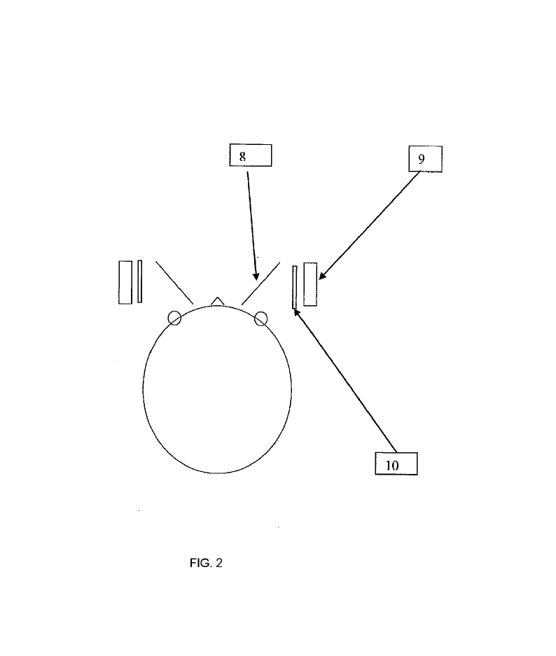

The subject or subjects 7 to be tested are fitted with a headset 5 which includes a plurality of electrodes for obtaining brain electrical activity from various sights on the scalp of the subject 7. In the event that the SSVER is used, the recording electrodes in the headset 5 are not used and a commercial MEG recording system such as the CTF MEG System manufactured by VSM MedTech Ltd. of 9 Burbidge Street, Coquitlam, BC, Canada, can be used instead. The headset includes a visor 4 which has for the left and right eyes of the subject half silvered mirrors 8 and white light Light Emitting Diode (LED) arrays 9, as shown in FIG. 2. The half silvered mirrors 8 are arranged to direct light from the LED arrays 9 towards the eyes of the subject 7. The LED arrays 9 are controlled so that the light intensity therefrom varies sinusoidally as a function of time under the control of control circuitry 6. The control circuitry 6 includes a waveform generator for generating the sinusoidal signal. In the event that the SSVER is used, the light from the LED array is conveyed to the visor via a fibre optic system. The circuitry 6 also includes amplifiers, filters, analogue to digital converters and a Universal Serial Bus (USB) interface or a Transmission Control Protocol (TCP) interface or other digital interface for coupling the various electrode signals into computer 1.

A translucent screen 10 is located in front of each LED array 9. Printed on the screen is an opaque pattern. The opacity of the opaque pattern is a maximum in a circular area in the center of the screen. Beyond the circular area, the opacity falls off smoothly with radial distance from the circular area circumference, preferably, the opacity should fall off as a Gaussian function described by Equation 8. The screen therefore reduces the flicker in the central visual field thus giving subjects a clear view of the visually presented material. The size of the central opaque circle (not shown) should be such as to occlude the visual flicker in the central visual field between 1-4 degrees vertically and horizontally. The translucent screen 10 impedes the flicker from the fovea of the subjects. The video screen 3 typically subtends an angle of 10-14 degrees vertically and horizontally as measured from the eyes of the subject.

If r<R then P=1;

If r≧R then P is given by Equation 8 below.

$$P = {e}^{- {( r - R )}^{2} / {G}^{2 }}$$

where:

P is the opacity of the pattern on the translucent screen;

R is the radius of the central opaque disk;

r is the radial distance from the center of the opaque disk; and

G is a parameter that determines the rate of fall-off of opacity with radial distance.
Equation 8

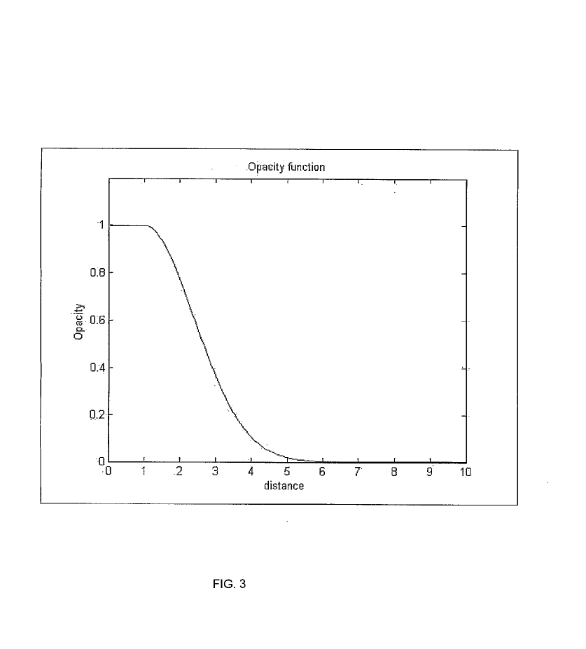

An opacity of P=1.0 corresponds to no light being transmitted through the screen while an opacity of P=0 corresponds to complete transparency. Typically G has values between R/4 and 2R. FIG. 3 illustrates the fall-off of opacity with radial distance from the center of the disk. In FIG. 3, R=1 and G=2R. While a Gaussian fall-off of opacity with radius is preferable, any function that is smooth and has a zero gradient at r=R and at r>3G is suitable.

The computer 1 includes software which calculates SSVEP or SSVER amplitude and phase and/or coherence from each of the electrodes in the headset 5 or MEG sensors.

Details of the hardware and software required for generating SSVEP and SSVER are well known and need not be described in detail. In this respect reference is made to the aforementioned United States patent specifications which disclose details of the hardware and techniques for computation of SSVEP. Briefly, the subject 7 views the video screen 3 through the special visor 4 which delivers a continuous background flicker to the peripheral vision. The frequency of the background flicker is typically 13 Hz but may be selected to be between 3 Hz and 50 Hz. More than one flicker frequency can be presented simultaneously. The number of frequencies can vary between 1 and 5. Brain electrical activity will be recorded using specialized electronic hardware that filters and amplifies the signal, digitizes it in the control circuitry 6 where it is then transferred to the computer 1 for storage and analysis. SSPT may also be used to ascertain regional SSVEP or SSVER phase increase at the scalp sites using SSPT analysis software, which is known and does not need to be described herein.

When using the SSVEP, brain electrical activity is recorded using multiple electrodes in headset 5 or another commercially available multi-electrode system such as Electro-cap (ECI Inc., Eaton, Ohio USA). When using the SSVER, commercial MEG recording system such as the CTF MEG System manufactured by VSM MedTech Ltd may be used. The number of electrodes or magnetic recording sites is normally not less than 8 and normally not more than 128, typically 16 to 32.

As mentioned above, the visor 4 includes LED arrays 9. In one embodiment, the light therefrom is varied sinusoidally. An alternative approach utilizes pulse width modulation where the light emitting sources are driven by 1-10 Khz pulses and the pulse duration is proportional to the brightness of the light emitting sources. In this embodiment, the control circuitry 6 receives a digital input stream from the computer 1 and outputs pulse width modulated pulses at a frequency of 1-10 Khz. The time of each positive going zero-crossing from the sinusoidal stimulus waveform or combination of stimulus waveforms is preferably determined to an accuracy of about 10 microseconds and stored in the memory of the computer 1.

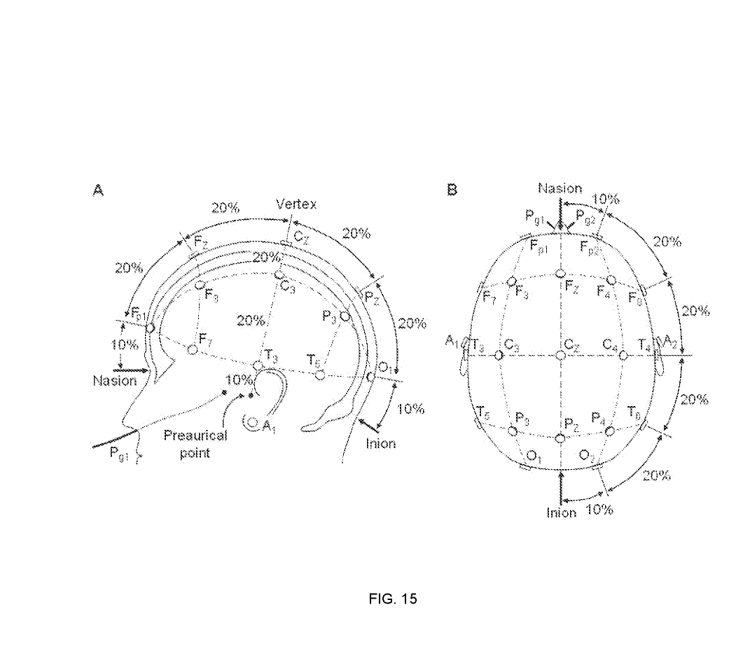

Brain electrical activity is recorded using outputs from the multiple electrodes in headset 5 or another commercially available multi-electrode system such as Electro-cap (ECI Inc., Eaton, Ohio USA). The number of electrodes is normally not less than 8 and normally not more than 128, typically 16 to 32. The electrodes are disposed so as to obtain outputs from selected scalp sites which are identified by the International 10-20 System of electrode locations shown schematically in FIG. 15.

Brain electrical activity at each of the electrodes is conducted to a signal conditioning system and control circuitry 6. The control circuitry 6 includes multistage fixed gain amplification, band pass filtering and sample-and-hold circuitry for each channel. Amplified/filtered brain activity is digitized to 16-24 bit accuracy at a rate not less than 300 Hz and transferred to the computer 1 for storage on hard disk. The timing of each brain electrical sample together with the time of presentation of different components of the audio-visual material are also registered and stored to an accuracy of 10 microseconds. The equivalent MEG recording system that is commercially available performs the same functions.

SSVEP and SSVER amplitude and phase can be calculated in accordance with the above.

While one or more subjects are viewing the images to be evaluated, the visual flicker is switched on in the visor 4 and brain electrical activity is recorded continuously on computer 1.

At the end of the recording stage, the SSVEP or SSVER amplitude and phase are separately calculated for each individual. Once all recordings are completed, group averaged data is calculated by averaging the smoothed SSVEP or SSVER amplitude and phase data from subjects to be included in the group (e.g. male, female, young, old, etc.).

### Method to Determine the Psychological Impact of Entertainment or Individual Presenters

At present, the likely success of newly created entertainment material such as television programs, feature films, and video games, or the response to an individual presenting a message, or to an individual seeking public office, is typically estimated by questionnaires with test audiences or focus groups drawn from test audiences that have viewed the material or the individual. Such methods are now recognized as deficient in tapping the emotional responses of the test audiences. It is such emotional responses such as the level of engagement with the material or individual, the sense of excitement, the likeability of various characters that play a crucial role in the commercial success or otherwise of the entertainment material.

SSVEP or SSVER phase increase is measured while subjects view an individual addressing an audience or some entertainment material. The entertainment material could comprise an episode from an established television program or a newly created pilot program. The material could also be presented in the form of an animatic, or a story board.

In one embodiment, the procedure to evaluate an established program or a newly developed pilot episode of a program is described as follows: 1. Individuals drawn from the target group or likely audience for the program view one or two episodes of the entertainment program. 2. On the following day, SSVEP or SSVER phase increase is measured while subjects view the next episode of the program.

In the situation where only animatics or story boards are available, SSVEP or SSVER phase increase is measured while subjects view the animatic or story board.

To determine the likely popularity of a completed program or early material, the most important measure is that of Engagement, as depicted in Equations 4 and 5.

The Engagement measure can also be used to estimate the likely popularity of program ideas when they are presented to a test audience in the form of animatics or story boards. Higher engagement when subjects view the animatic or story board will be associated with a higher likelihood that the finished program will be popular with the test audience.

Audience response either to an individual or to various characters in the entertainment material can also be estimated from SSVEP or SSVER phase increase. Greater audience acceptance of an individual or an actor is indicated by higher engagement when that actor is featured.

The likeability or the extent to which the individual or actor is liked by the audience is indicated by the Attraction-Repulsion measure. A positive value for the attraction measure is associated with the participants finding the character or individual attractive and liked while a negative measure is associated with repulsion or dislike.

The memorability or extent to which an actor's role is encoded in long-term memory is dependent upon Long Term Memory encoding for details and verbal memories associated with an actor's role. This is indicated by SSVEP phase increase at left frontal region, preferably approximately equidistant from left hemisphere electrodes C₃, F₃ and F₇ at the time that the actor is featured. If inverse mapping techniques are used, the relevant location in the left cerebral cortex is the vicinity of Brodmann areas 6, 44, 45, 46 and 47.

The pre-determined psychological state of Long Term Memory encoding for emotional and non-verbal memories associated with an actor's role is indicated by SSVEP phase increase at right frontal region, preferably approximately equidistant from left hemisphere electrodes C₄, F₄ and F₈ at the time that the actor is featured. If inverse mapping techniques are used, the relevant location in the right cerebral cortex is the vicinity of Brodmann areas 6, 44, 45, 46 and 47.

The emotional excitement associated with a speech given by an individual or a program or a scene in a program is given by the Emotional Intensity measure, indicated by SSVEP or SSVER phase increase at the right parieto-temporal region, preferable approximately equidistant from right hemisphere electrodes O₂, P₄ and T₆. If inverse mapping techniques are used, the relevant location in the right cerebral cortex is the vicinity of the right parieto-temporal junction.

The SSVEP or SSVER phase increase measures of Engagement, Attraction-Repulsion and Emotional Intensity can also be used to select the most suitable performer or actor for a given role. In this case, an audience would view each of the applicants for a part performing a given scene in a program. The actor eliciting the highest level of Engagement and Likeability (on the Attract-Repulsion score) would be the most suitable one for the role. In the case of an individual giving an election speech or a presentation, the measures of Engagement, Attraction-Repulsion and Emotional Intensity associated with different points made in the speech would enable identification of the issues that elicit the strongest responses in the audience. The issues that elicit the strongest responses are thus those that have the greatest impact on the wider audience.

This method of evaluating entertainment material can also be used with different media such as entertainment delivered to a computer over the internet or entertainment delivered to a mobile phone or other digital media.

### Example 1

The following procedure is used to evaluate the likely success of new entertainment material or the release of established entertainment material to a new target audience.

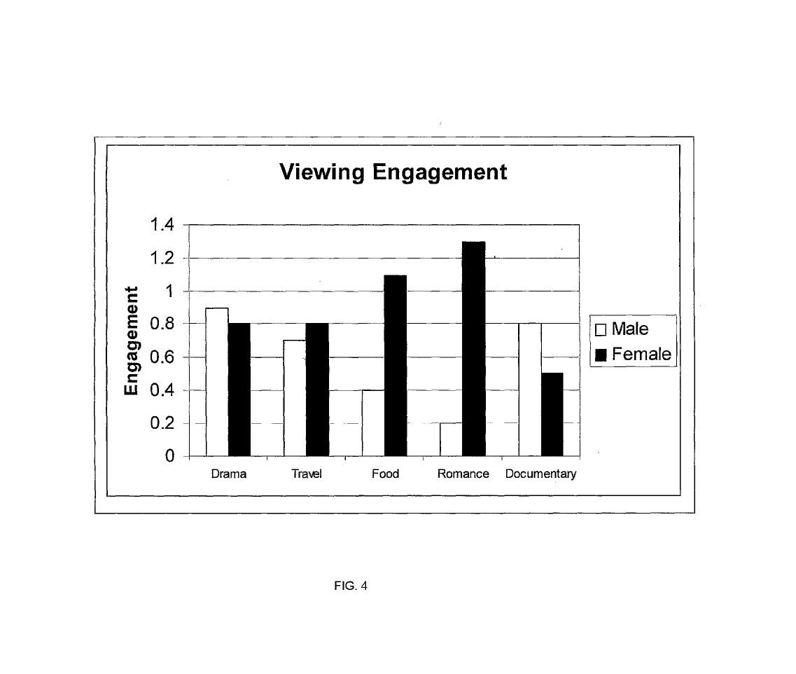

In this example, 50 to 200 participants drawn from the likely target audience for the test entertainment material are recruited into the study. All participants view at least one episode or part of the entertainment material at either one or more locations or in the home. In this Example, viewer engagement is important and accordingly the electrodes in the headsets 5 are selected so as to enable engagement to be calculated using the techniques described earlier. Brain activity is preferably determined using SSVEP or SSVER phase increase. The engagement measures of the target audiences were separated into males and females and the results were plotted graphically in FIG. 4. FIG. 4 shows the engagement measures for the male and female audiences for five different types of programs, drama, travel, food, romance and documentary. Later, ideally no less than 24 hours later, SSVEP or SSVER phase increase is recorded while the participants view a subsequent episode or part of the entertainment material as described in more detail below.

To record SSVEP or SSVER phase increase, a selected number of subjects, say 50, are seated in a test room and the headsets 5 are placed on their heads. The visors 4 are then placed in position and adjusted so that the foveal block by the screens 10 prevents the appearance of the flicker over the screens 3 where the images are presented. The number of subjects in a recording session is variable and typically can vary from 1 to over 100. When pooling subjects to create the average response, the number of subjects whose data is to be included in the average should preferably be no less than 16.

To minimize irritation or discomfort to the participants due to the flicker, the flicker stimulus is of variable intensity and only switched to the highest intensity when material of interest to the client such as particular segments of the program or specific actors appear on the screen. During the periods that material of interest is not present on the screen, the stimulus intensity is typically zero and never more than 10% of the typical value used when material of interest is on the screen. Preferably, the stimulus is not switched on abruptly but is slowly increased before the segment of interest is displayed and decreased slowly after the end of the material of interest. Typically, the stimulus is increased linearly over a 30-60 second epoch prior to the appearance of the material of interest so that it reaches its maximum value 60 seconds prior to the appearance of the material of interest. At the end of every segment of interest, a 30 second sequence of still images of scenery and a musical accompaniment is presented. Typically, 60 images are presented over the period of 30 seconds with each image present for about 0.5 seconds. SSVEP or SSVER phase increase levels during the adjacent scene images are used as a reference level for SSVEP or SSVER phase increase during the preceding segment of interest. This enables removal of any long-term changes in SSVEP or SSVER phase increase that may occur over the time course of the recording period.

Immediately the sequence of reference images at the end of the segment of interest, the stimulus intensity is linearly reduced to the minimum value over a 30 second period. The slow linear increase and decrease of stimulus intensity occurs for every segment of interest.

The likely audience engagement is given by the brain engagement measure time averaged over at least 5 minutes of a typical segment of the new entertainment material, engagement being calculated separately for males and females using the SSVEP techniques described above. As can be seen, for males, the programs with the highest engagement, and hence the greatest likelihood of success are the drama and documentary programs while for the females audience, the romance and food programs are most likely to be successful.

### Example 2

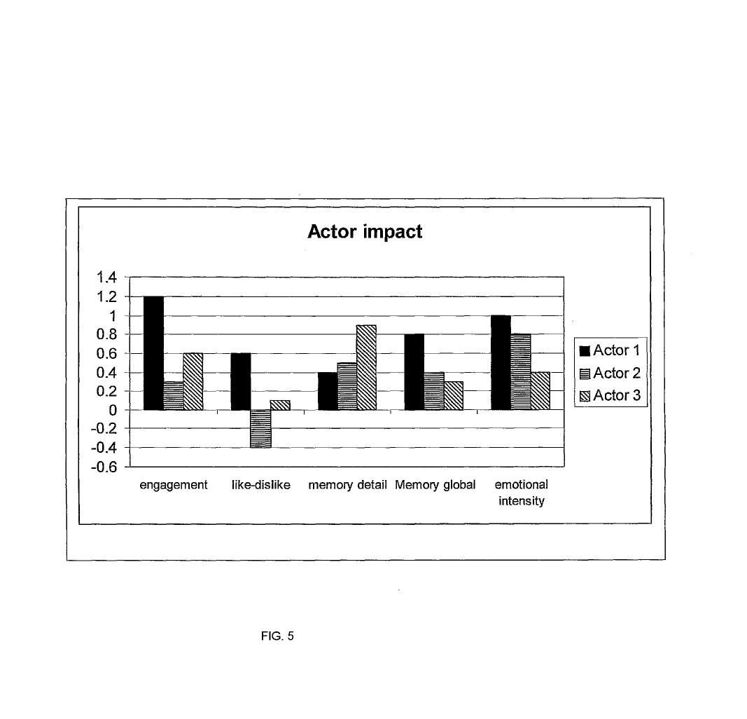

The invention can also be used to determine the psychological impact of various actors which are featured in entertainment material. This example is similar to Example 1 except that it is not necessary that the target audience has viewed an earlier episode of the entertainment material. Also the electrodes are selected so as to enable assessment of engagement, like-dislike, memory for detail and verbal features, memory for non-verbal features and emotion, and emotional intensity. Again, SSVEP or SSVER phase increases are preferably measured using SSVEP or SSVER techniques. In this example, a segment of entertainment material has three different actors, Actor 1, Actor 2 and Actor 3 featured therein. Pooled responses are plotted graphically in FIG. 5 for the various hypothetical measures. It is apparent from FIG. 5 that Actor 1 scores high on engagement, likeability and emotional intensity. This indicates that the audience is able to identify with the Actor 1 (indicated by high engagement), likes the actor (high likeability) and finds the actor exciting (high emotional intensity). By contrast, Actor 2 is disliked and also arouses strong emotion. This actor could be a good choice to play the part of a villain. Finally, Actor 3 is modestly engaging and the details of his role are well remembered (high memory for detail). Actor 3 could be well suited to educational roles where content is more important.

### Example 3

The invention can also be used to select an actor for a specific role. In this application, each of the possible actors is required to read the same script or act the same role. SSVEP or SSVER phase increase is then recorded from the test audience while viewing each of the applicants for the given role. Depending on the nature of the role (e.g. hero, villain, etc.) the actor most effectively eliciting the desired psychological response would be selected for the part. Most relevant measures for the central characters would be engagement, like-dislike, and emotional intensity. If the role also has an educational or information transfer component, long-term memory encoding would also be important.

It will be appreciated by those skilled in the art that the method of the invention compares very favourably with known techniques for evaluating the likely commercial success of entertainment material, suitability of actors or suitability of persons for public office. In the case of entertainment material, known analytical techniques can be used to determine a behavioural measure such as a Q-Score. The Q-Score indicates the desire the average viewer feels about watching a particular program. Typically, the Q-Score is only available for programs where a number of complete episodes have been viewed by the target audience. In the case of new entertainment material, this would be quite time consuming and expensive to produce. By contrast, the assessment techniques based on engagement measures give an indication of the popularity based on the pilot program which of course is relatively inexpensive to produce. FIG. 6 illustrates the average level of engagement multiplied by 100 estimated from an audience of 150 subjects over a five minute period when watching three television programs, sport, drama and travel. The level of engagement measured from SSVEP or SSVER phase increase in accordance with the invention is shown in solid black bars. Corresponding data obtained from known Q-Score techniques are plotted in striped bars. It will be seen that there is a strong correlation between the techniques of the invention and the Q-Score results, notwithstanding that the techniques of the invention have been based on a pilot programs.

Many modifications will be apparent to those skilled in the art without departing from the spirit and scope of the invention.

### Assessment of Computer Games

The likely success of any game depends on the extent to which the player is engaged in the game and also the extent to which particular situations elicit the desired emotional state, such as excitement, fear, pleasure, etc.

The most important psychological measure is ‘engagement.’ The extent to which the game engages the player is given by is given by the weighted mean SSVEP or SSVER phase increase during the initial period at prefrontal sites described by Equation 4. If inverse mapping techniques are used, the relevant expression is described in Equation 5.

Other psychological measures and their SSVEP or SSVER phase increase indicators that are of relevance include Visual Attention, Emotional Intensity, and Attraction-Repulsion.

Visual Attention associated with a given set of situation parameters is indicated by increased SSVEP or SSVER phase increase at left and right occipital recording sites. In the International 10-20 system that labels recording sites on the brain, the positions referred to above correspond to the vicinity of O₁ and O₂. If activity in deeper parts of the brain are assessed using inverse mapping techniques such as BESA, EMSE or LORETA in combination with either electrical or magnetic recordings or SSVEP or SSVER, the relevant location in the left cerebral cortex is the vicinity of the left and right occipital lobe (Brodmann area 17).

The Emotional Intensity, associated with a set of situation parameters is indicated by increased SSVEP or SSVER phase increase at right parieto-temporal region, preferable approximately equidistant from right hemisphere electrodes O₂, P₄ and T₆ during the initial period. If inverse mapping techniques are used, the relevant location in the right cerebral cortex is the vicinity of the right parieto-temporal junction.

The extent to which individuals are attracted or repelled by a game situation associated with a given set of situation parameters is given by the difference between SSVEP or SSVER phase increase at left frontal/prefrontal and right frontal/prefrontal regions. Attraction is indicated by a larger activity in the left hemisphere compared to the right, while repulsion is indicated by greater activity in the right hemisphere compared to the left, as depicted in Equation 6. If inverse mapping techniques are used, the relevant expression is depicted in Equation 7.

### Determining Computer Game Situation Parameters

The game situation parameters are a set of digital values that uniquely identify the situation of the game player. These parameters will vary with the nature of the game and will also vary with time as the player progresses through the game. For instance, in a driving simulation game, the game situation parameters could comprise the location of the player's car on the simulated track or landscape, the speed and direction of the player's car as well as the state of the steering wheel, brakes and gears. In an adventure game, the game situation parameters may include the location and orientation of the player's representation (avatar) within the simulated environment such as a building, battleground or streetscape. In addition, the game situation parameters could include the status of the avatar such as its capabilities (e.g., strength, ‘magical powers,’ etc.) as well as the location and actions of other avatars (in multi-player games) or computer generated denizens such as monsters, aliens, wizards, etc. The game situation parameters change with time and a record of each game situation parameter as a function of time can be stored as a numerical array in the game computer memory. While a game is being played, the relevant game situation parameters are held in computer memory and when active playing ceases transferred to hard disk memory or another digital storage medium such as flash memory.

The game software developers would use standard software such as C++ or specialized computer games development software such as DaskBASIC (The Game Creator Ltd, ‘Rockville,’ Warrington Rd, Lower Ince, Wigan, Lancashire, WN3 4QG, UK) to incorporate the software to identify and store the game situation parameters while a game is being played.

It will be appreciated that the present invention provides a method that relies on measurement of SSVEP or SSVER phase increase rather than verbal responses to questionnaires or other voluntary feedback in order to determine an individual player's response to various components of a computer game. Accordingly, the method of the invention enables game developers to improve the likely commercial success of the game by modifying components of the game that are found to be less engaging.

In one embodiment, SSVEP or SSVER phase increase is measured while subjects or players take part in the computer game. Simultaneously, the specific situations encountered by the player are also recorded as a stream of digital parameters specifying the player situation or Situation Parameters.

FIG. 1 schematically illustrates a system 50 for determining the response of a subject or player to a computer game presented on a video screen 3 and loudspeaker 2.

Typically, 20 to 100 players will play the game while SSVEP or SSVER phase increase and Situation Parameters are recorded. To determine the SSVEP or SSVER phase increase associated with a specific set of situation parameters or a range of situation parameters, individual player SSVEP or SSVER phase increase is averaged for all points in time where the recorded situation parameters satisfy certain predetermined criteria. For each individual player, this will yield a set of mean SSVEP or SSVER phase increase measures associated with each of the situation parameter criteria. SSVEP or SSVER phase increase for a given situation parameter criterion is then averaged across all the players or subset of players.

While participants are playing the computer game, the visual flicker is switched on in the visor 4 and brain electrical activity is recorded continuously on the computer 1, as described herein. At the end of the recording stage, the SSVEP or SSVER amplitude and phase are separately calculated for each individual, as described herein.

### Example 4

In the following example, a computer game development company needs to assess the psychological impact of a computer game under development. 20 to 100 participants drawn from the target market for the game are recruited into the study. SSVEP or SSVER phase increase is then recorded while the participants play the computer under development. Each participant plays the game on an individual computer located in a booth to reduce distraction. To record SSVEP or SSVER phase increase, the headsets 5 are placed on their heads and the visors 4 are placed in position and adjusted so that for each participant the foveal block by the screens 10 prevents the appearance of the flicker over the central portion of the screen 3.

Once SSVEP or SSVER phase increase and situation parameters have been recorded for all game playing participants, each participant's SSVEP or SSVER phase increase is averaged when the situation parameters satisfy certain criteria. As an example, one such criterion could be a specific geographical location and speed prior to a collision in a racing car game. Alternatively, in a war game, it could be a particular battlefield location when the player is under attack from more than three enemy soldiers. Each game would therefore have a unique set of situation parameters criteria that reflected the components of the game where the game developer required player psychological information. SSVEP or SSVER phase increase measured for the various situation parameters criteria can then be averaged across all the players to obtain a representative response for each criterion or set of specified situation parameters.

While the most important psychological parameters are engagement and attention, other parameters may also be important at various portions of the game. For example, emotional intensity may be important in certain components of the game while long-term memory may be important where information needs to be remembered or where advertising takes place in the game. The psychological parameters can be measured using the techniques described earlier and these can be plotted graphically for the various game situation parameters of interest. The game developer can then determine which of the game parameters has a relatively low entertainment value. These parts of the game could therefore be eliminated or modified to make them more interesting so as to achieve higher measures of engagement and attention or other psychological responses of interest.

The accuracy of the assessment can be improved by measuring the SSVEP or SSVER phase increase of the players against reference levels. One convenient way to do this would be to average the SSVEP or SSVER phase increase for each player during the whole game and then compare the SSVEP or SSVER phase increase during the game situations of interest to the average game level. This provides a more accurate measure of the players' psychological responses to the game situations of interest. Alternatively, prior to commencement of a game, each of the players could be presented with a series of still images or the like together with musical accompaniment SSVEP or SSVER phase increases measured in the usual way during this reference period. SSVEP or SSVER phase increases can then be assessed against the reference levels, which also provides increased accuracy. Reference periods presented in this way also provide an opportunity for comparisons to be made between game situations of different games rather than game situations within a single game.

Many modifications will be apparent to those skilled in the art without departing from the spirit and scope of the invention.

### Method to Evaluate Psychological Responses to Visual Objects

SSVEP or SSVER phase increase and eye position can be combined to indicate the psychological response associated with visual attention to specific components of the visual image or product. This would enable advertisers, manufacturers, web site developers and architects the opportunity modify and hence improve the visual material such as text, billboard, product, building or web site. These will be collectively termed ‘visual objects’ in the description which follows.

SSVEP or SSVER phase increase and gaze position are simultaneously measured while subjects view any type of visual display such as a page of text, an advertising billboard, object, a product such as a car or a perfume bottle or a building or a part of a building. The image may comprise an object, a display on a video monitor or a ‘virtual reality’ display. The term “visual display” is intended to encompass all of the foregoing.

To determine the SSVEP or SSVER phase increase associated with a specific visual image and gaze location, individual subject SSVEP or SSVER phase increase is averaged for all points in time when the gaze position is in the vicinity of a specific part of the image. For each subject, this will yield a set of mean SSVEP or SSVER phase increase measures associated with a specific part of the image. SSVEP or SSVER phase increase for a given part of the image is then averaged across all the subjects or subset of subjects. The likely effectiveness of the image or product depends on the extent that the image elicits the desired emotional or cognitive state.

In the event that the image constitutes a billboard or print advertisement, the key psychological measures are the levels of attention, the strength of the emotional response and the extent to which the key messages are encoded in long-term memory.

If the image constitutes an object or product such as an item of furniture, a car or a view of a room, the key psychological measures may be Engagement, Attention, Desirability, Emotional Intensity and Attraction.

Visual attention associated with an image or part of an image is indicated by increased SSVEP or SSVER phase increase at left and right occipital recording sites. In the International 10-20 system that labels recording sites on the brain, the positions referred to above correspond to the vicinity of O₁ and O₂. If activity in deeper parts of the brain are assessed using inverse mapping techniques such as BESA, EMSE or LORETA in combination with either electrical or magnetic recordings or SSVEP or SSVER, the relevant location in the left and right cerebral cortex is the vicinity of the left and right occipital lobe (Brodmann area 17).

In particular, the desirability associated with the image or part of an image of a product is indicated by increased SSVEP or SSVER phase increase at left and right parietal recording sites during the initial period. In the International 10-20 system that labels recording sites on the brain, the positions referred to above correspond to the vicinity of P₃ and P₄. If activity in deeper parts of the brain are assessed using inverse mapping techniques such as BESA, EMSE or LORETA in combination with either electrical or magnetic recordings or SSVEP or SSVER, the relevant location in the left and right cerebral cortex is the vicinity of the left and right intraparietal area.

The emotional intensity associated with an image or product or a component of an image or product is indicated by increased SSVEP or SSVER phase increase at right parieto-temporal region, preferable approximately equidistant from right hemisphere electrodes O₂, P₄ and T₆ during the initial period. If inverse mapping techniques are used, the relevant location in the right cerebral cortex is the vicinity of the right parieto-temporal junction.

How well various parts of the text or images are stored or encoded in long-term memory is indicated by increased SSVEP or SSVER phase increase at left and right temporal sites in the vicinity of T₅ and T₆ and also at right frontal sites equidistant between C₄, F₄ and F₈, and also at left frontal sites equidistant between C₃, F₃ and F₇ during the initial period. If inverse mapping techniques are used, the relevant locations in the left and right temporal lobes in the vicinity of Brodmann area 20 and in the left and right frontal cortex in the vicinity of Brodmann areas 6, 44, 45, 46 and 47.

The extent to which individuals are attracted or repelled by the various parts of the product image is given by the difference between SSVEP or SSVER phase increase at left frontal/prefrontal and right frontal/prefrontal regions. Attraction is indicated by a larger activity in the left hemisphere compared to the right while repulsion is indicated by greater activity in the right hemisphere compared to the left, as depicted in Equations 6 and 7. A positive value for the attraction measure is associated with the participants finding the image or product attractive and liked while a negative measure is associated with repulsion or dislike.

### Measuring Gaze Position

A number of techniques whose principles are in the public domain are available to measure gaze position. The most suitable for use in the method of the invention utilizes a commercially available system such as ‘TrackIR’ produced by Natural Point Inc, of Corvallis, Oreg. 97339, USA. This comprises an infra-red camera mounted on a helmet worn by the subject. The infra-red camera coupled with an infra-red landmarks near the visual display enable head position to be determined. Eye position within the orbit of the eye can be measured by infra-red oculography (Reulen, J. P. H. et al., “Precise Recording of Eye Movement: the IRIS Technique Part 1,” and “Stimulation and Recording of Dynamic Pupillary Reflex: the IRIS Technique Part 2,” Medical and Biological Engineering and Computing, 26(1988): 20-32). Commercial systems to measure eye position such as the Skalar Iris Limbus Tracker are available from Cambridge Research Systems Ltd., 80 Riverside Estate, Sir Thomas Longley Road, Rochester, Kent ME2 4BH England. Infra-red oculography lends itself best to the use of the steady state visually evoked potential (SSVEP) as the infra-red light emitting diodes and photo-transistors can be incorporated into the SSVEP visor. Combining head position information derived from the camera with eye position from the infra-red oculography enables one to determine gaze position. Using infra-red oculography system in combination with the TrackIR head position system enables gaze position to be determined to an accuracy of 0.25 degrees and updated every 40 msec.

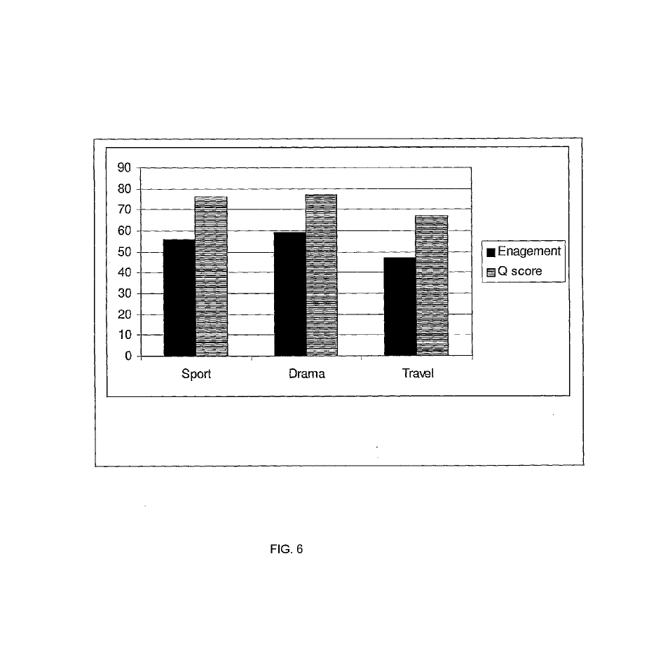

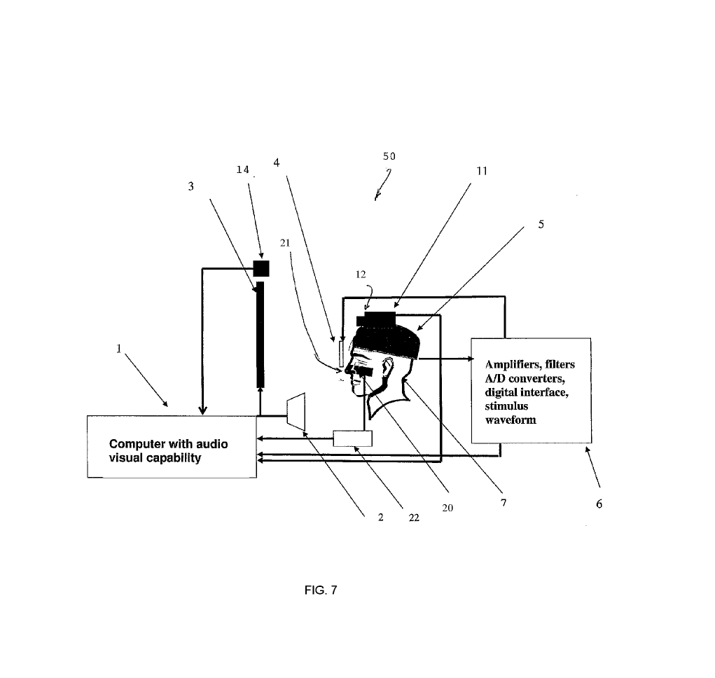

FIG. 7 schematically illustrates a system 50 for determining the response of a subject or player to a computer game presented on a video screen 3 and loudspeaker 2. The system includes a computer 1 which controls various parts of the hardware and also performs computation on signals derived from the SSVEP or SSVER phase increase of the subject 7, as will be described below. The computer 1 also presents the computer game which can be presented to the subject 7 on the screen 3 and/or through the loudspeaker 2.

The subject 7 to be tested are fitted with a headset 5 which includes a plurality of electrodes for obtaining brain electrical activity from various sights on the scalp of the subject 7. The system includes a head tracking system 12 which preferably is the TrackIR head position tracking system referred to above and includes a head mounted camera 11, cables connecting the camera 11 to the computer 1 and software running on the computer 1.

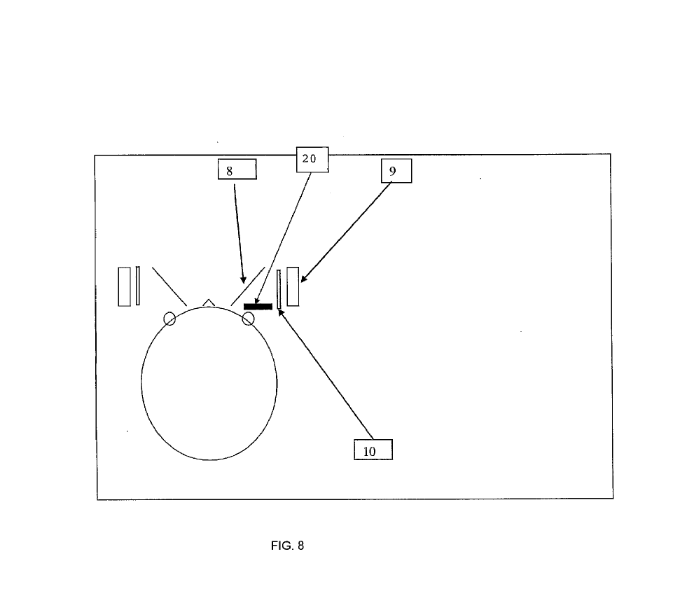

FIG. 9 schematically illustrates the operation of the head tracking system 12 in more detail. The system includes an infra-red light reference source 14 which produces at least two beams 30 and 32 of infra-red radiation. The beams are oriented at predetermined directions relative to one another and are generally directed at the subject 7. The head mounted camera 11 receives components of the two beams depending on the orientation of the head of the subject and from this information, the supplied software can compute the position of the head relative to the screen 3. The output from the camera 11 is coupled to the computer 1 and the software is arranged to sample the video output from the camera 11 at a predetermined sampling rate, say 20 times per second, in order to provide adequate temporal resolution of the position of the subject's head relative to the screen 3.

In the event that the SSVER is used, the recording electrodes in the headset 5 are not used and a commercial MEG recording system such as the CTF MEG System manufactured by VSM MedTech Ltd of 9 Burbidge Street Coquitlam, BC, Canada, can be used instead. The headset includes a visor 4 which includes half silvered mirrors 8 and white light Light Emitting Diode (LED) arrays 9, as shown in FIG. 8.

The half silvered mirrors 8 are arranged to direct light from the LED arrays 9 towards the eyes of the subject 7.

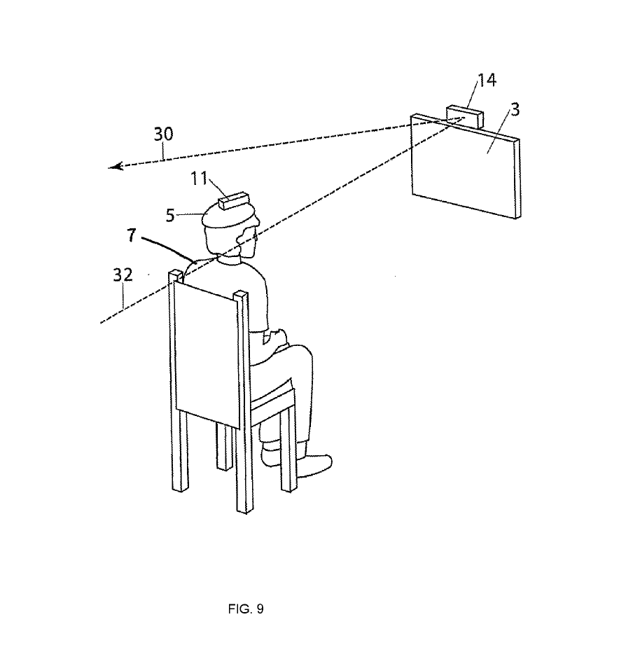

The system 50 also includes an oculography or eye tracking system 21 which is used to track the position of the subject's left or right eye so that this information combined with the output from the head position tracking system can be used to accurately determine the position of the gaze of the subject 7 relative to the center of the screen 3. The eye tracking system 21 may be the scalar Iris Limbus Tracker referred to above. Briefly, the eye tracking system 21 includes an infra-red sensor assembly 20 and signal processing circuitry 22. The infra-red sensor assembly 20 is mounted on the headset 5 adjacent to the eye of the subject 7, as schematically indicated in FIGS. 7 and 8. FIG. 10 shows the details of the an infra-red sensor assembly 20 in more detail. It will be seen that it includes an infra-red LED 16 mounted above the eye 23 of the subject 7 and a photo-transistor 17 which is sensitive to infra-red located beneath the eye 23. The LED 16 directs an infra-red beam at the lateral edge of the cornea 19 and sclera 18 border, the photo-transistor also being arranged to detect reflected infra-red light from this area. The photo-transistor 17 is coupled to provide input signals to the signal processing circuitry 22 which functions as an interface for the computer 1.

The gaze position as a function of time is calculated from the head position information supplied by the head tracking system (TrackIR) system 12 and the eye tracking system 21. Gaze position measurements are calibrated for each subject 7 prior to the evaluation of a visual display. This is done by displaying a small target on the screen 3, such as a cross or a small circle at five locations in succession. These are the center of the screen and the four diagonals of the screen, i.e., top left, top right, bottom left and bottom right. In each case, the target is located for 1 to 5 seconds in each location, preferably 1 second. This sequence is repeated twice. In the first instance, subjects are instructed to initially look directly ahead and not move their head as they follow the target with their eyes. During the second sequence, subjects are asked to follow the target by moving their head and not moving their eyes.

From these two sets of measurements, it is a straight forward task to calculate gaze location from the outputs of the head position and oculography systems.

The gaze position is determined by summing the relevant spherical polar coordinates available from the head position and oculography system 21. This is given by the following equations:

Θ_{gaze}=Θ_{head position}+Θ_{oculography} Equation 9

Φ_{gaze}=Φ_{head position}+Φ_{oculography}Equation 10

In the preferred system 50, the LED arrays 9 are controlled so that the light intensity therefrom varies sinusoidally under the control of control circuitry 6. The control circuitry 6 includes a waveform generator for generating the sinusoidal signal. In the event that the SSVER is used, the light from the LED array is conveyed to the visor via a fibre optic system. The control circuitry 6 also includes amplifiers, filters, analogue to digital converters and a USB interface or a TCP interface or other digital interface for coupling the various electrode signals into the computer 1.

SSVEP and SSVER amplitude and phase are calculated as disclosed herein. Amplitude and phase components can be calculated using either single cycle Fourier coefficients (aₙ and bₙ) or coefficients that have been calculated by smoothing across multiple cycles (Aₙ and Bₐ). Equations 2 and 3 describe the procedure for calculating the smoothed SSVEP or SSVER coefficients for a single subject. For pooled data, the SSVEP or SSVER coefficients (Aₙ and Bₙ) for a given electrode are averaged (or pooled) across all of the subjects or a selected group of subjects.

While one or more subjects are viewing the images to be evaluated, the visual flicker is switched on in the visor 4 and brain electrical or magnetic activity is recorded continuously on the computer 1.

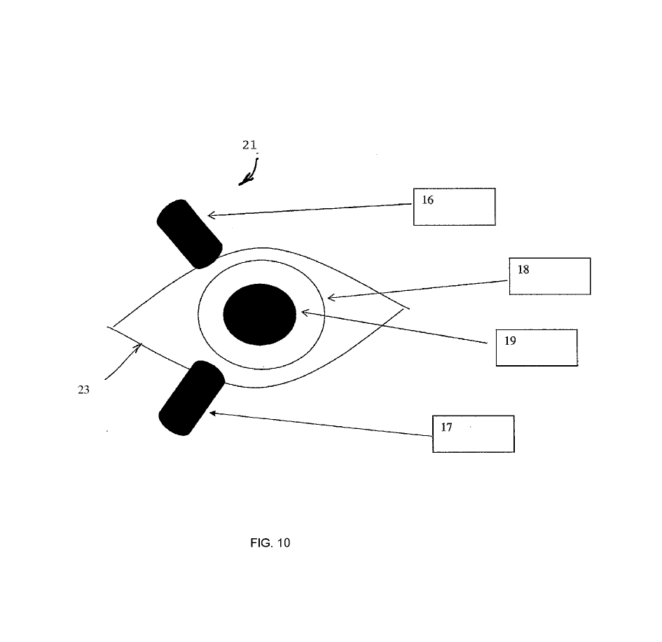

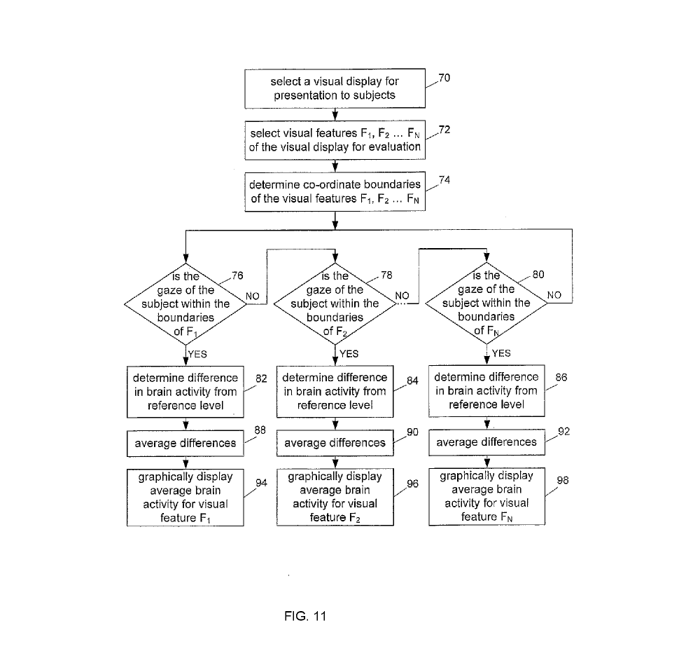

FIG. 11 is a simplified flowchart showing a typical sequence of steps used in the method of the invention. The flowchart includes an initial step 70 in which the customer selects a visual display which is to be evaluated by the method of the invention. After the initial step, step 72 indicates the selection by the customer of the particular visual features F₁, F₂ . . . Fₙ of the visual display which are to be evaluated. The method then moves to step 74 in which the boundaries of the visual features F₁, F₂ . . . Fₙ are determined and these are then preferably expressed in terms of spherical polar coordinates, the datum of which is the center of the screen 3. The method then moves to first question box 76 which determines whether the gaze of the subject, as determined by the head tracking system 12 and eye tracking system 21, is within the coordinate boundaries of visual feature F₁. If not, the method turns to a second question box 78 which determines a similar question with respect to the boundaries of visual feature F₂ and so on until the final question box 80 determines whether the gaze is within the boundaries of visual feature Fₙ. If no, then the sequence returns to the first question box 76 as shown.

If the gaze is within the boundary of visual feature F₁, then the software in the computer 1 determines the difference in SSVEP or SSVER phase increase from the reference level as indicated by step 82. The result is then accumulated in a running average step 88 and, at the end of the display sequence, step 94 indicates a graphical display of the average SSVEP or SSVER phase increase for visual feature F₁.

Similarly, where the gaze of the subject is determined to fall within the boundaries of the visual feature F₂, as determined by the second question box 78, the software determines the SSVEP or SSVER phase increase differences in step 84, the moving average in step 90 and generates the display in step 96. Similarly, if the gaze is determined to fall within the boundaries of visual feature Fₙ, as determined by question box 80, the software determines the difference in SSVEP or SSVER phase increase from the reference in step 86, the moving average in step 92 and generates the graphical display in step 98.

It will be appreciated that steps 82, 84 and 86 can be determined from different scalp sites so as to measure difference psychological responses, such as emotional responses, attention, long term memory encoding, engagement, desirability and likeability as described above. The various psychological responses are not separately shown for clarity of illustration. They can, however, be averaged and graphically displayed if required.

Further, the SSVEP or SSVER phase increase can be determined in various ways, as indicated herein, including gamma or high frequency EEG or MEG activity; frequency of EEG or MEG alpha activity; or SSVEP or SSVER amplitude and phase measurements.

Where SSVEP or SSVER phase increase is determined by measuring SSVEP or SSVER, the amplitude and phase are preferably separately calculated for each subject at the end of the recording stage. Once all recordings are completed, group averaged data associated with specific gaze locations on the test object is calculated by averaging the smoothed SSVEP or SSVER amplitude and phase data from subjects to be included in the group (e.g., male, female, young, old, etc.) for different gaze locations on the test object. Separate group averages associated with predetermined gaze locations on the test object may then be calculated.

### Example 5

Each subject 7 is seated before a video monitor and the headset 5 is placed on the subject's head. The visor 4 is then placed in position and adjusted so that the foveal block by the screens 10 prevents the appearance of the flicker over the screens 3 where the visual objects are presented. The head tracking system 12 and the eye tracking system 21 are then initialized, in accordance with the procedures described above. When pooling subjects to create the average response, the number of subjects whose data is to be included in the average should preferably be no less than 16.

Visual objects appear on the screen for different periods of time. Print and outdoor display material can be presented for 5 to 300 seconds depending on the amount of text while products and packaging can be presented as either a still image or rotating on a platform for 10 to 180 seconds. Architectural objects such as buildings, building interior and outdoor structures can be viewed as still images or animated sequences where the viewer moves through a path in space, similar to virtual reality.

In a typical study, one or more visual objects are presented to the subjects in a sequence. Each sequence of visual objects lasting no more than 300 seconds is followed immediately by a 30 second reference period in which a sequence of still images of scenery and a musical accompaniment. Typically, 60 images were presented over the period of 30 seconds with each image present for 0.5 seconds. The same sequence of images and music were presented after each sequence of visual objects. SSVEP or SSVER phase increase levels during the adjacent scene images are used as a reference level for SSVEP or SSVER phase increase during the preceding visual objects. This enables removal of any long-term changes in SSVEP or SSVER phase increase that may occur over the time course of the recording period.

Pooled or averaged data at various brain sites associated with specific gaze locations on the test object can then be displayed to the client as the difference between the reference level and the value when participants are viewing specific locations on the visual object. A fixed offset between 0.2 to 0.6, preferably 0.3 radians is then added to the abovementioned difference to yield the SSVEP phase data at each scalp site.

FIG. 12 shows the visual object to be tested in accordance with the method of the invention. In this case, the visual object is a perfume bottle 100 having a main body 102, label 104, neck 106 and stopper 108. The purpose of the study was to determine the level of attractiveness of the bottle and to see what parts of the bottle are viewed more favourably than others. In this case, the perfume bottle 100 is selected to have two visual features for evaluation. The first visual feature is the upper part of the bottle which includes the neck 106 and stopper 108. The operator determines the boundary 110 of these visual features using standard software packages such as PowerPoint (Microsoft Corporation, One Microsoft Way, Redmond, Wash. 98052, USA) or CorelDraw (Corel Corporation, 1600 Carling Avenue, Ottawa, Ontario K1Z 8R7, Canada) and these are stored in the memory of the computer 1. A second part of the image of the object is then selected for evaluation. In this case it is the main body 102 of the bottle and the boundaries are determined, as indicated by boundary line 112. The coordinates of the boundary line 112 are entered in the memory of the computer 1, as before.

The display sequence is presented to the subjects 7 and the SSVEP or SSVER phase increases are measured and recorded, in accordance with the procedures described above and the results plotted, as described below.

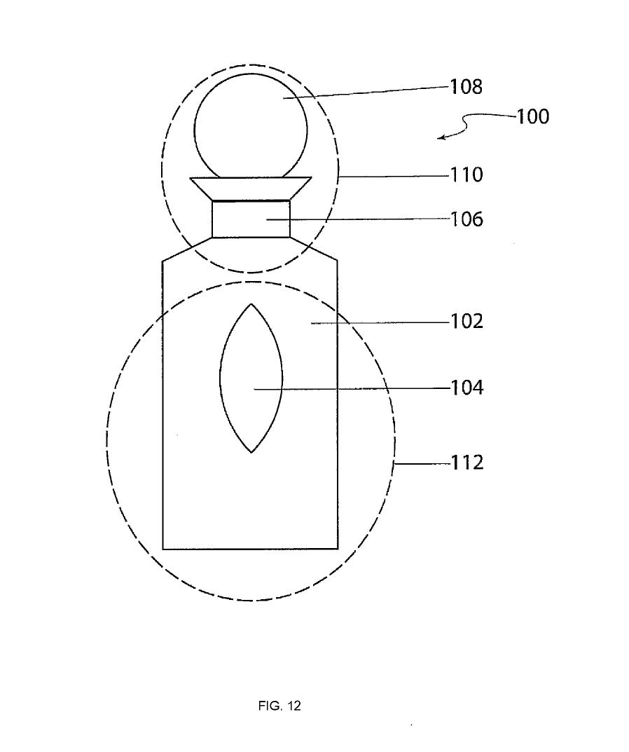

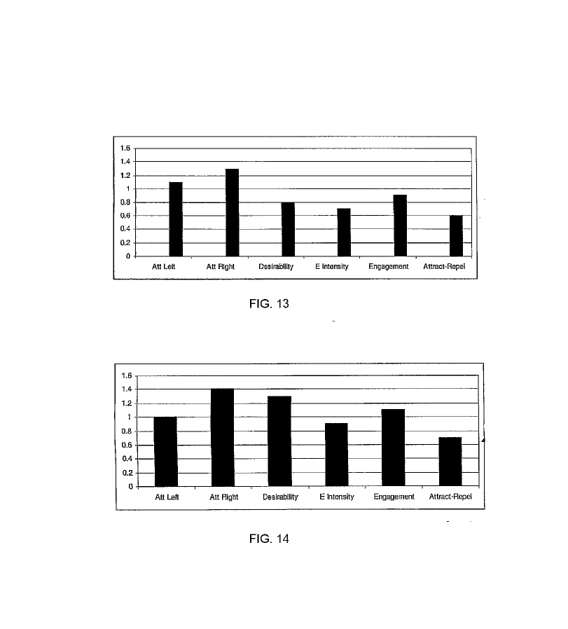

FIG. 13 shows the SSVEP or SSVER phase increase for the upper part of the bottle which includes the neck 106 and stopper 108. It will be seen from FIG. 13 that there are relatively high levels of global attention (associated with aesthetic judgments), engagement and desirability.

FIG. 14 graphically illustrates activity associated with the main body 102 of the bottle. It will be seen that FIG. 14 shows that there is elevated levels of global attention and desirability.

In this example, the client would be advised that the design is attractive to the target audience and that the body of the bottle is especially attractive. Any changes to the specific design of this bottle should avoid those regions already considered attractive and desirable.

Many modifications will be apparent to those skilled in the art without departing from the spirit and scope of the invention.

### Method for Evaluating the Effectiveness of Commercial Communication

Over the last 20 years, neuroscience researchers have learnt more about the specialized role of different brain regions associated with specific psychological states or processes. By measuring the activity in different brain regions while participants perceive advertisements, it is possible to infer the psychological state elicited by the advertisement.

One embodiment of the invention relates to a method and system to identify the psychological state experienced by the subject while perceiving an advertisement. SSVEP or SSVER phase increase is measured using a technique termed Steady State Probe Topography (SSPT). A method is disclosed that uses SSPT to determine the cognitive and emotional states that are elicited by the advertisements and in particular, particular points in time during the advertisement. Effectiveness is then defined in terms of whether the psychological states elicited by the advertisements are those intended by the creator of the advertisement. In particular, the most likely measure of effectiveness is the level of memory encoding during the branding component of the advertisement, i.e. when the brand is displayed during the advertisement.

Reference is made to articles entitled “Brain-Imaging Detection of Visual Scene Encoding in Long-Term Memory for TV Commercials,” Rossiter, J. R. et al., March/April 2001 Journal of Advertising Research, pages 13-21 and “Frontal Steady-State Potential Changes Predict Long-Term Recognition Memory Performance,” by Silberstein, R. B. et al., International Journal of Psycho Physiology 39 (2000): 79-85, the content of these articles are incorporated herein by cross-reference. The first article describes an experiment using steady state probe topography (SSPT), which is a technique that involves presenting a visual or auditory or combined task such as an advertisement whilst subjects view a peripheral flickering light. In the experiment, subjects were tested for long term memory recall of static frames from TV commercials presented to the subjects one week after viewing the commercials. The article suggests that recall of static frames from the commercial by subjects provides a measure of evaluation of the success of the TV commercial.

It has now been appreciated that the effectiveness of commercial communications or advertising can be evaluated by analysis of responses in different regions of the brain so as to identify predetermined psychological states elicited by the participants as they perceive the commercial communication or advertisement.

Key psychological measures of pre-determined psychological states include Visual Attention to Detail, Visual Attention to Global Features, Multi-Modal Attention to Detail (Desirability), Multi-Modal Attention to Global Features (Desirability), Emotional Intensity, Attraction-Repulsion, Engagement, and Behavioral Intent. The expression does not include long term memory encoding per se because long term memory encoding is not considered to be a psychological state for the purposes of the methods of assessment described herein.

FIG. 1 schematically illustrates a system 50 for determining the response of a subject or a group of subjects to audiovisual material presented on a video screen 3 and loudspeaker 2. The system includes a computer 1 which controls various parts of the hardware and also performs computation on signals derived from the SSVEP or SSVER phase increase of the subject 7, as will be described below. The computer 1 also holds the television program and advertisements which can be presented to one or more subjects 7 on the screen 3 and/or through the loudspeaker 2.

Once the system has been set up and headsets 5 fitted to one or more subjects, the audiovisual program or advertisement to be evaluated is displayed on the screen 3, the visual flicker is switched on in the visor 4 and brain electrical activity of the subject or subjects is recorded continuously on the computer 1. At the end of the recording stage, the SSVEP or SSVER amplitude and phase are separately calculated for each subject. Once all recordings are completed, group averaged data is calculated by averaging the smoothed SSVEP or SSVER amplitude and phase data from selected subjects to be included in the group (e.g., male, female, young, old). Changes in regional synaptic excitation or inhibition are indicated by SSVEP or SSVER phase changes while changes in regional activity (irrespective of whether these changes are associated with excitation or inhibition) are indicated by changes in SSVEP or SSVER amplitude. Typically, such inverse mapping techniques require 19 or more scalp recording sites and preferably 64 or more scalp recording sites.

In accordance with the invention, cognitive and emotional measures at specific points in time during the advertisement can be derived from the pattern of SSVEP or SSVER phase and SSVEP or SSVER amplitude changes at the various scalp recording sites or inferred brain regional activity. Scalp recording sites typically used are identified by reference to the International 10-20 System (Jasper, H. H., Electroenceph. Clin. Neurophysiol., 10(1957): 370-5) shown in FIG. 15. The psychological states associated with SSVEP or SSVER phase and amplitude changes at scalp and brain cortical locations are summarized herein.

Behavioral Intent, or the likelihood that the subjects are likely to act in accordance with the purpose of the screened advertisement, that is make a purchase, is associated with memory encoding at the time of branding. This is indicated by the level of long term memory encoding at the time that the brand is portrayed in the advertisement.

Once data from long term memory encoding has been obtained for verbal and/or non-verbal memories, this can be correlated with the time sequence of the advertising material being presented to determine the relevant levels of memory encoding when the brand is portrayed in the advertisement. After this correlation, it is then possible to assess whether or not the subjects are likely to purchase goods or services shown in the advertisement.

Activity at various brain sites can be determined using a variety of possible inverse mapping techniques such as BESA, EMSE and LORETA that express brain activity in selected brain regions in terms of a function of SSVEP or SSVER amplitude and phase measures recorded over the entire scalp. Typically such techniques require more than 20 scalp recording sites and preferably 64 or more scalp recording sites.

### Example 6

The following procedure is used to evaluate a television advertisement to a client such as the brand manager of a company and/or advertising agency.

A selected number of subjects, say 50, are seated in a test room and the headsets 5 are placed on their heads. The visors 4 are then placed in position and adjusted so that the foveal block by the screens 10 prevents the appearance of the flicker over the screens 3 where the advertisements are presented. The number of subjects in a recording session is variable and typically can vary from 1 to over 100. When pooling subjects to create the average response, the number of subjects whose data is to be included in the average should be no less than 16.

The advertisements to be tested were included in an ‘advertising break’ comprising a block of 3 to 6 advertisements incorporated in a television program to simulate a standard commercial TV viewing environment. Each advertising break is followed immediately by a 30 second sequence of still images of scenery and a musical accompaniment. Typically, 60 images were presented over the period of 30 seconds with each image present for 0.5 seconds. The same sequence of images and music were presented after each advertisement break. SSVEP or SSVER phase increase levels during the adjacent scene images are used as a reference level for SSVEP or SSVER phase increase during the preceding advertisement break. This enables removal of any long-term changes in SSVEP or SSVER phase increase that may occur over the time course of the recording period.

Pooled or averaged data at various brain sites can then be displayed to the client as the difference between the reference level and the value at other points in time during the advertisement. A fixed offset between 0.2 to 0.6, preferably 0.3 radians is then added to the abovementioned difference to yield the SSVEP phase data at each scalp site.

To minimize subject irritation or discomfort to the subject due to the flicker, the flicker stimulus is of variable intensity and only switched to the highest intensity when material of interest to the client such as the block of advertisements is present on the screen. During the periods that material of interest is not present on the screen, the stimulus intensity is typically zero and never more than 10% of the typical value used when material of interest is on the screen. Preferably, the stimulus is not switched on abruptly but is slowly increased before the advertisement break and decreased slowly after the end of an advertisement break. Typically, the stimulus is increased linearly over a 30-60 second epoch prior to the advertisement break so that it reaches its maximum value 60 seconds prior to the first advertisement. Immediately the sequence of reference images at the end of the advertisement break ends, the stimulus intensity is linearly reduced to the minimum value over a 30 second period. The slow linear increase and decrease of stimulus intensity occurs for every advertisement break.

FIG. 17 schematically illustrates the television advertisement to be assessed. The advertisement is in relation to a sports utility vehicle 30. The vehicle 30 is being driven along a road 32 in a dark forest 34 on a wet and stormy night, as indicated by still segment a. The vehicle 30 drives through a partially flooded roadway so as to cause a large spray 36 from the vehicle, as indicated in still segment b. The vehicle is then shown traversing steep grade, as shown in still segment c. The driver 38 then stops the vehicle where a large log 40 has fallen on the road 32 so as to remove it, as shown in still segment d. After clearing the branch from the road, the driver is about to re-enter the car when he realizes that he is covered with mud and would soil the interior of the car were he to enter immediately, as indicated in still segment e. This scene features an abrupt change in the soundtrack when the music stops and is replaced by a period of silence. The driver ruefully finds a cake of soap in the glove compartment and proceeds to take a bath in the nearby stream during the storm. The final segment of the animated part of the advertisement shows a block of text with the brand of the vehicle in the text block, as indicated by still segment f. The final segment g runs for about 2 seconds and shows the brand and product being advertised.

Once the final subject recording has taken place, individual SSVEP or SSVER data is pooled for each specific group of subjects, for example, separate pooling or averaging could be effected for the entire group and also for subgroups such as male and female and young and old. To display the SSVEP or SSVER phase data in relationship to the associated commercial, an in-house program (NV-Show) is used. NV-Show displays the advertisement in a portion of a computer video monitor and the associated SSVEP or SSVER phase data as a graph below. As the advertisement is played in the upper portion of the screen, the graph describing the SSVEP or SSVER phase data is revealed below. Alternatively, the graph of SSVEP or SSVER phase data can be present throughout the advertisement playing time and a moving time marker used to indicate the SSVEP or SSVER phase data corresponding to time in the advertisement being displayed.

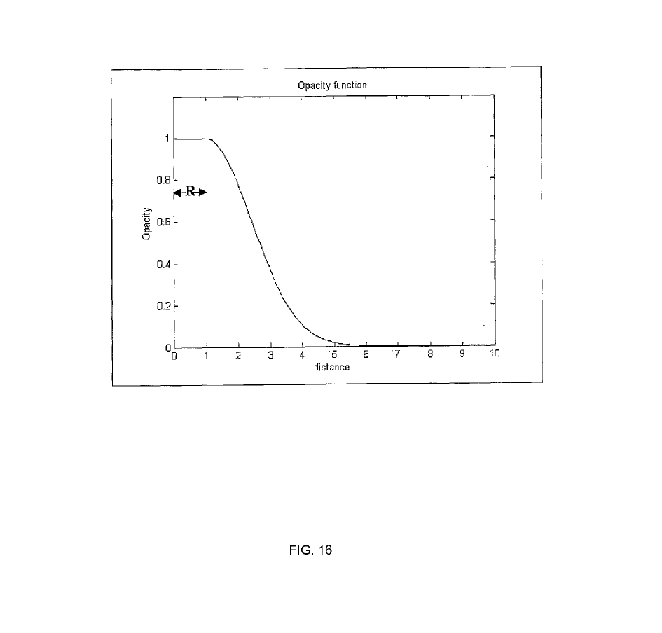

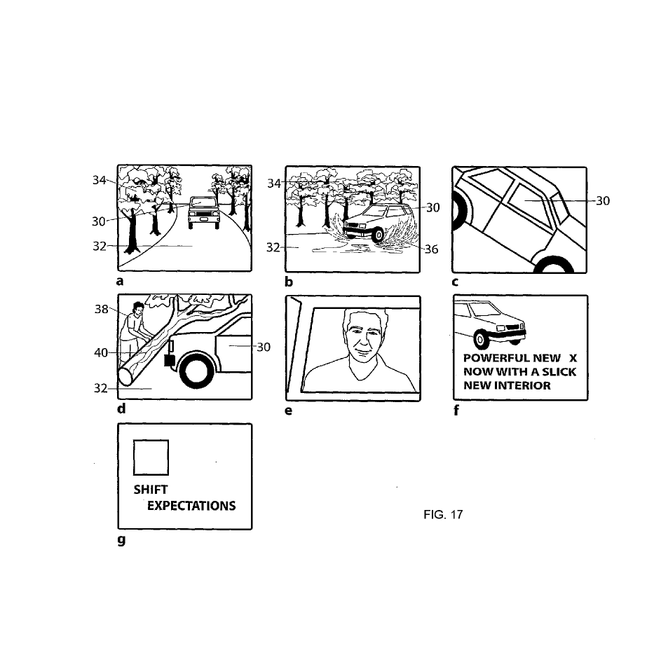

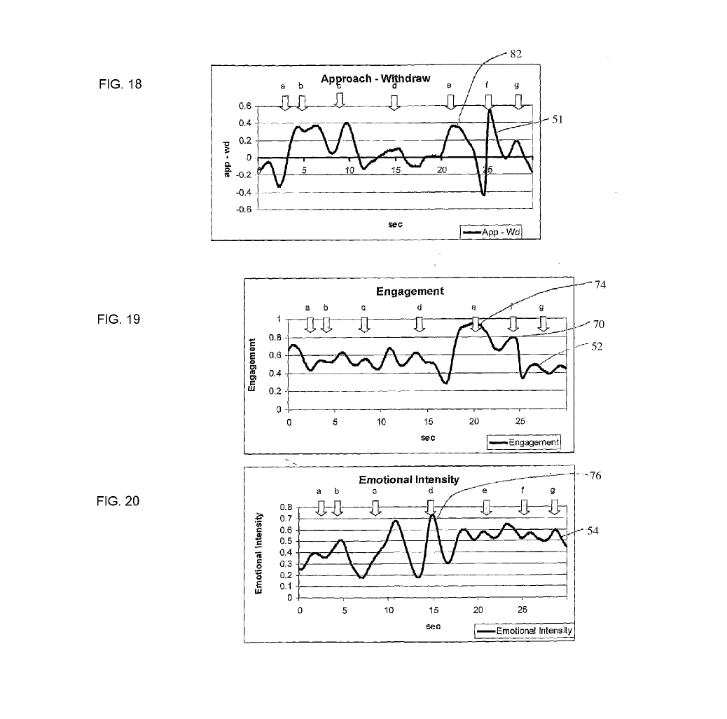

FIGS. 18 to 23 illustrate the brain measurement values at the various brain sites during the advertisement. The block arrows labeled ‘a’ to ‘g’ in FIGS. 18 to 23 correspond to the points in time a to g shown in FIG. 17.

In FIG. 18, the line 51 shows the Attraction-Repulsion measure during the advertisement. The Attraction-Repulsion measures are calculated using Equation 6 above and the results are displayed in graphical form on a time scale which corresponds to the duration of the advertisement. Positive values or values above the horizontal time axis correspond to ‘approach’ or ‘like’ while negative values correspond to points in time where the audience ‘withdraws’ or ‘dislikes.’ For the approach-withdraw measure in this study, any value 0.3 or <−0.3 is statistically significant.

If a greater number of electrodes were used, it would be possible to use inverse mapping techniques to obtain somewhat higher resolution in the data and by using Equation 7.

In FIG. 19 the line 52 indicates the level of audience Engagement in the advertisement as a function of time. The data is calculated using Equation 4 and is again presented graphically on a time scale which corresponds to that of the advertisement. The term Engagement relates to the level of personal or emotional relevance experienced by the subjects. In the study illustrated, Engagement must exceed 0.4 to be significantly greater than baseline level while an engagement level above 0.7 is considered high. If inverse mapping techniques are used, Equation 5 can be used to calculate the measure for Engagement.

In FIG. 20 the line 54 indicates the equivalent changes in Emotional Intensity elicited by the advertisement. Emotional Intensity refers to the intensity of emotion experienced by the subjects irrespective of the type of emotion, e.g. fear, joy, anger, excitement, etc. The data can be calculated as described above by using SSVEP or SSVER phase increase at right parieto-temporal region, from an electrode which is approximately equidistant from the right hemisphere electrodes O₂, P₄ and T₆. If inverse mapping techniques are used, the relevant location in the right cerebral cortex is in the vicinity of the right parieto-temporal junction. Once again, any level above 0.4 is significantly higher than background while a level above 0.7 is considered high.

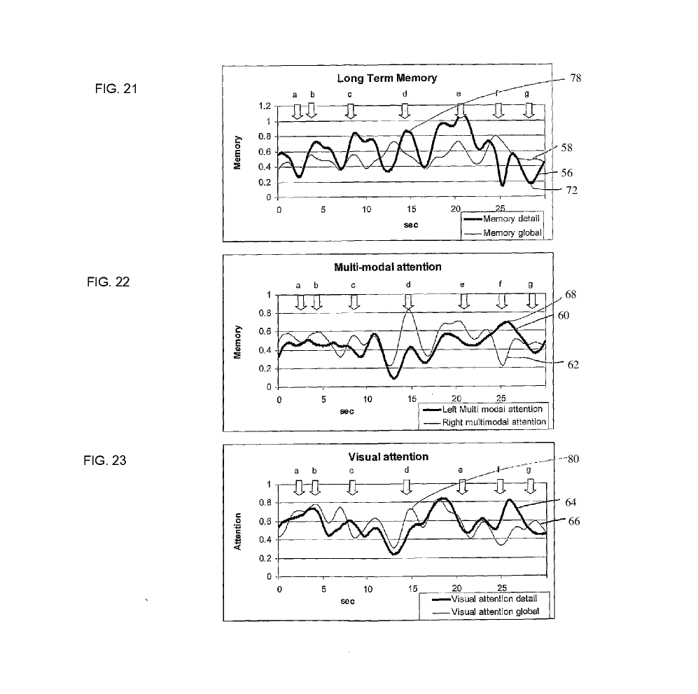

The lines 56 and 58 in FIG. 21 indicate memory encoding in detail and memory encoding global respectively during the advertisement. This measure indicates how effectively different components of the advertisement are being stored in long term memory. This measure is especially important as an advertisement is only effective if the key messages and information about the brand enters long-term memory. It has been found that the likely impact of the advertisement on Behavioural Intent is positively correlated with the level of long-term memory encoding during the time that branding information is presented in the advertisement. Thus Behavioural Intent or the likely impact on behaviour of the subjects is indicated by the level of memory encoding at the time that branding information is presented in the advertisement, i.e., at points f and g where the branding information is positively displayed during the advertisement. Both memory traces are important in regard to Behavioural Intent. As long as either left or right memory encoding state is high during “branding,” Behavioural Intent or the propensity to act on the message of the advertisement is high.

In FIG. 22 the lines 60 and 62 indicate left hemisphere multi-modal attention and right hemisphere multi-modal attention respectively. Multi-modal attention includes visual and auditory attention to details of the advertisement being displayed to the subjects. Specifically, the line 60 indicates attention to detailed features such as text and speech and the line 62 indicates attention to global features such as facial expression and music (for example).

In FIG. 23 the lines 64 and 66 indicate the visual attention to detail and visual attention globally of the subjects to the advertising material being displayed. Specifically, the line 64 indicates visual attention to detailed features such as text. The line 64 indicates visual attention to global features such as facial expression (for example).

### Assessment

For this advertisement to be assessed as effective, it is necessary for memory encoding to be high during the times when the product or product benefits are emphasized, such as at point f and when the brand is explicitly featured such as at point g. As can be seen in FIG. 22, left memory encoding is moderate to high during point f as indicated by peak 68 in the line 60 indicating that there is adequate memory encoding for the text information THE POWERFUL NEW X; NOW WITH A SLICK NEW INTERIOR near the end of the advertisement. During this point in time, Engagement is also high indicating a high degree of personal relevance regarding the features mentioned as indicated by the peak 70 in line 52.

By contrast, the memory encoding for the branding information at point g is low and Engagement is also low for this time as indicated by trough 72 in the line 56. This indicates that the subjects will remember the vehicle interior but will not recall the brand. The Behavioural Intent measure is also low indicating that the advertisement is not effective as the subjects will be less inclined to act in accordance with the advertisement. This is again indicated by the trough 72.

Accordingly, the advertisement can be assessed as not being commercially effective in that the branding information is not satisfactorily encoded in long-term memory. The dramatic structure of the advertisement is, however, effective in that ‘joke’ at point e in the advertisement works because the scene where the driver realizes he can't re-enter the car without washing is well encoded in long-term memory and also leads to highest level of Engagement as indicated by the peak 74 in line 52. In addition, there is a high level of Attraction that is associated with this humor as indicated by peak 82 in line 51.

It is also apparent that the overall structure of the advertisement is effective in that the key dramatic scene where the driver has to get out of the car to clear the road at point d is associated with high levels of Emotional Intensity as indicated by the peak 76 in line 54; high levels of memory encoding as indicated by peak 78 in line 56; and a high level of Attention to Global Features as indicated by the peak 80 in line 66.

After assessment of the advertisement using the techniques of the invention, the client can appreciate that the overall effect of the advertisement is positive but some modification is required at points f and g where the brand is introduced. Accordingly, the client is in a position to modify the advertisement so that it should become highly successful and avoid incurring substantial advertising costs in an advertising program which does not induce viewers to purchase the product.

It will be appreciated that the principles of the invention can be applied other audiovisual commercial communications in addition to advertisements.

Many modifications will be apparent to those skilled in the art without departing from the spirit and scope of the invention.

Method to Determine Attributes Associated with a Brand or Product

Preferably, SSVEP or SSVER phase increase is measured while subjects view a brand image or a product image, represented by the brand name and logo on a video display or a visual presentation of the product and product name. The brand or product image remains on the screen for an initial duration of 0.5 sec to 5 sec, preferably, 1 sec. During a subsequent period, the brand or product image remains on the screen and a word describing a quality or semantic probe appears under the brand or product image. The duration of the subsequent period is 0.5 sec to 5 sec, preferably the same duration as the initial period.

Brand image and product image need not be static and a moving product image or brand image may be used also. The semantic probe is most commonly one or more words but may also be a sound or another image.

The psychological response to the brand or product image alone can be ascertained from the distribution of SSVEP or SSVER phase increase during the initial period when the brand or product image is presented. This measure refers to the level of Visual Attention to Detail or text elicited by the brand or product image during the initial period.

SSVEP or SSVER phase increase at the left occipital region, preferably electrode O₁ in the International 10-20 system has been found to be relevant to assessment of the subject's Visual Attention to Detail. If inverse mapping techniques are used, the relevant location in the left cerebral cortex is the vicinity of Brodmann area 17.

SSVEP phase increase at the right occipital region, preferably electrode O₂ in the International 10-20 system has been found to be relevant to the assessment of the subject's Visual Attention to Global Features including responses to facial expressions displayed on the screen. If inverse mapping techniques are used, the relevant location in the right cerebral cortex is the vicinity of Brodmann area 17.

The Multi-Modal Attention (desirability) associated with the brand or product image is indicated by increased SSVEP or SSVER phase increase at left and right parietal recording sites during the initial period. In the International 10-20 system that labels recording sites on the brain, the positions referred to above correspond to the vicinity of P₃ and P₄. If inverse mapping techniques are used, the relevant location in the left cerebral cortex is the vicinity of the left and intraparietal areas.

The Emotional Intensity associated with the brand or product image is indicated by increased SSVEP or SSVER phase increase at right parieto-temporal region, preferable approximately equidistant from right hemisphere electrodes O₂, P₄ and T₆ during the initial period. If inverse mapping techniques are used, the relevant location in the right cerebral cortex is the vicinity of the right parieto-temporal junction.

How well the brand image or product image is stored or encoded in Long Term Memory is indicated by increased SSVEP or SSVER phase increase at left and right temporal sites in the vicinity of T₅ and T₆ and also at right frontal sites equidistant between C₄, F₄ and H during the initial period. If inverse mapping techniques are used, the relevant locations in the left and right temporal lobes in the vicinity of Brodmann area 20 and in the right frontal cortex in the vicinity of Brodmann areas 6, 44, 45, 46 and 47.

The extent to which individuals are attracted or repelled by the brand image or product image is given by the difference between SSVEP or SSVER phase increase at left frontal/prefrontal and right frontal/prefrontal regions. Attraction is indicated by a larger activity in the left hemisphere compared to the right while Repulsion is indicated by greater activity in the right hemisphere compared to the left, as depicted in Equations 6 and 7.

The extent of Engagement of the brand image or product image is given by the weighted mean SSVEP or SSVER phase increase during the initial period at prefrontal sites expressed in Equations 4 and 5.

The association between specific characteristics and the brand or product image is indicated by the distribution of SSVEP or SSVER phase increase during the appearance of the brand or product image and the semantic probe. Congruence between the semantic probe and the qualities associated with the brand or product image in the mind of the subjects is indicated by increased SSVEP or SSVER phase increase at right prefrontal sites, in the vicinity of electrode Fₚ₂. If inverse mapping techniques are used this corresponds to SSVEP or SSVER phase increase at right orbito-frontal cortex (in vicinity of Brodmann area 11). Incongruence between the semantic probe and the qualities associated with the brand or product image in the mind of the subjects is indicated by reduced brain at right prefrontal sites, in the vicinity of electrode F_{N}. If inverse mapping techniques are used, these correspond to reduced SSVEP or SSVER phase increase at right orbito-frontal cortex (in vicinity of Brodmann area 11).

FIG. 1 schematically illustrates a system for determining the response of a subject or a group of subjects to audio-visual material presented on a video screen 3 and loudspeaker 2. The system includes a computer 1 which controls various parts of the hardware and also performs computation on signals derived from the SSVEP or SSVER phase increase of the subject 7, as will be described below. The computer 1 also holds the images and sounds which can be presented to one or more subjects 7 on the screen 3 and/or through the loudspeaker 2.

A translucent screen 10 is located in front of each LED array 9. Printed on the screen is an opaque pattern. The opacity is a maximum in a circular area in the center of the screen as shown in FIG. 16. Beyond the circular area, the opacity falls off smoothly with radial distance from the circular area circumference, preferably, the opacity should fall off as a Gaussian function described by Equation 8. The screen reduces the flicker in the central visual field thus giving subjects a clear view of the visually presented material. The size of the central opaque circle should be such as to occlude the visual flicker in the central visual field between 1 to 4 degrees vertically and horizontally.

R is the radius of the central opaque disk while r is the radial distance from the center of the opaque disk. G is a parameter that determines the rate of fall-off of opacity with radial distance. Typically G has values between R/4 and 2R. FIG. 16 illustrates the fall-off of opacity with radial distance from the center of the disk. In FIG. 16, R=1 and G=2R.

The computer 1 includes software which calculates SSVEP or SSVER amplitude and phase from each of the electrodes in the headset 5 or MEG sensors.

Details of the hardware and software required for generating SSVEP and SSVER are well known and need not be described in detail. In this respect reference is made to the aforementioned United States patent specifications which disclose details of the hardware and techniques for computation of SSVEP. Briefly, the subject 7 views the video screen 3 through the special visor 4 which delivers a continuous background flicker to the peripheral vision. The frequency of the background flicker is typically 13 Hz but may be selected to be between 3 Hz and 50 Hz. More than one flicker frequency can be presented simultaneously. The number of frequencies can vary between 1 and 5. Brain electrical activity will be recorded using specialized electronic hardware that filters and amplifies the signal, digitizes it in the control circuitry 6 where it is then transferred to the computer 1 for storage and analysis.

When using the SSVEP, brain electrical activity is recorded using multiple electrodes in headset 5 or another commercially available multi-electrode system such as Electro-cap (ECI Inc., Eaton, Ohio USA). When using the SSVER, commercial MEG recording system such as the CTF MEG System manufactured by VSM MedTech Ltd may be used. The number of electrodes or magnetic recording sites is normally not less than 8 and normally not more than 128, typically 16 to 32.

Brain electrical activity at each of the electrodes is conducted to a signal conditioning system and control circuitry 6. The control circuitry 6 includes multistage fixed gain amplification, band pass filtering and sample-and-hold circuitry for each channel. Amplified/filtered brain activity is digitized to 16-24 bit accuracy at a rate not less than 300 Hz and transferred to the computer 1 for storage on hard disk. The timing of each brain electrical sample together with the time of presentation of different components of the audio-visual material are also registered and stored to an accuracy 10 microseconds. The equivalent MEG recording system that is commercially available performs the same functions.

While one or more subjects are viewing the images to be evaluated, the visual flicker is switched on in the visor 4 and brain electrical activity is recorded continuously on the computer 1.

At the end of the recording stage, the SSVEP or SSVER amplitude and phase are separately calculated for each individual. Once all recordings are completed, group averaged data is calculated by averaging the smoothed SSVEP or SSVER amplitude and phase data from subjects to be included in the group (e.g. male, female, young, old, etc).

### Example 7

The following procedure is used to evaluate the brand attributes for a client. A selected number of subjects, say 50, are seated in a test room and the headsets 5 are placed on their heads. The visors 4 are then placed in position and adjusted so that the foveal block by the screens 10 prevent the appearance of the flicker over the screens 3 where the images are presented. The number of subjects in a recording session is variable and typically can vary from 1 to over 100. When pooling subjects to create the average response, the number of subjects whose data is to be included in the average should be no less than 16.

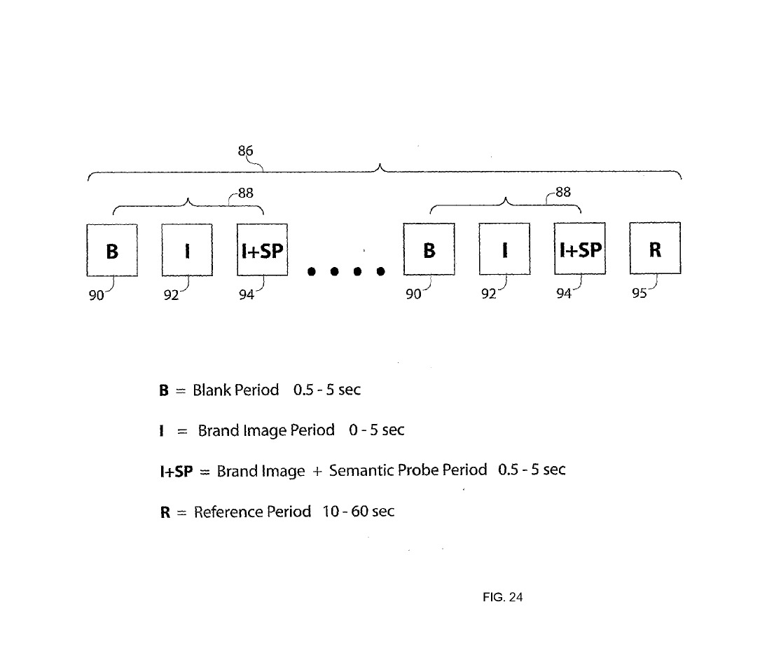

The brand or product images to be evaluated are presented to the subjects in a particular sequence. FIG. 24 diagrammatically illustrates a typical sequence 86. The sequence itself is made up of a number of blocks 88, each of which commences with a blank period 90, a brand image period 92 in which the brand or product image is displayed on the screen followed by a congruence period 94 in which the same brand or product image of that block 88 and a semantic probe are simultaneously displayed. In the illustrated arrangement, each of the blank periods 90, brand image periods 92 and congruence periods 94 are of the same length which is in the range from 0.5 to 5 secs. The full sequence 86 includes a reference period 95 which follows the last block 88. The reference period has a duration from 10 to 60 secs and preferably about 30 secs in which neutral material such as images of scenery are sequentially displayed. The reference period 95 preferably displays the images of scenery for 0.5 secs and has musical accompaniment.

The sequence 86 may include any convenient number of blocks 88. In a typical evaluation of a brand or product image, there may be three to six blocks 88 presented to the subjects in which different semantic probes are presented during the congruence periods 94. In addition, five to ten different brands may be included in the sequence 86. Accordingly, there may be from fifteen to sixty blocks 88 in the sequence 86.

SSVEP or SSVER phase increase is recorded from the subjects during each of the periods 90, 92 and 94 and reference SSVEP or SSVER phase increase is calculated for each subject during the reference period 95.

In a typical assessment, sequences 86 are incorporated into a television program. The first sequence 86 is typically presented early in the television program while the second sequence 86 is presented late in the program, after one or more ‘advertising breaks’ that may be included in the program. It is preferred that the advertising breaks are followed by a similar reference period 95. The reference period 95 preferably displays the images of scenery for 0.5 secs and has musical accompaniment. It is also preferred that the reference periods 95 are the same in the two sequences 86 and are the same after the advertising breaks. SSVEP or SSVER phase increase levels during the reference periods 95 are used as reference levels for SSVEP or SSVER phase increase during the preceding blocks 88 and the advertisement breaks. This enables removal of any long-term changes in SSVEP or SSVER phase increase that may occur over the time course of the recording period.

Pooled or averaged data at various brain sites can then be displayed to the client as the difference between the reference level and the value at other points in time during the sequence 86. A fixed offset between 0.2 to 0.6, preferably 0.3 radians is then added to the abovementioned difference to yield the SSVEP phase data at each scalp site.

In the sequence illustrated in FIG. 24, each of the blocks 88 commences with a blank period 90. This is thought to be highly preferable so as to properly distinguish SSVEP or SSVER phase increase levels between periods 92 and 94 of adjacent blocks 88. It is possible, however, to reduce the duration of the blank periods 90 to zero in which case this could be offset by making the duration of the blocks 92 and 94 much longer so as to enable adequate differentiation between the periods 92 and 94 in adjacent blocks 88.

It is also preferred to have the reference level 95 at the end of the sequence 86. This assists in obtaining a better reference level because if the reference period 95 were at the commencement of the sequence, then the subjects may have some initial interest in whatever material was initially presented and this might lead to somewhat inaccurate results. Where a number of sequences are included in a television program then it is probably less important that the reference period 95 be at the end of the sequences 86 for second and subsequent sequences 86.

To minimize subject irritation or discomfort to the subject due to the flicker, the flicker stimulus is of variable intensity and only switched to the highest intensity when material of interest to the client such as the sequence 86 or advertisement break is present on the screen. During the periods that material of interest is not present on the screen, the stimulus intensity is typically zero and never more than 10% of the typical value used when material of interest is on the screen. Preferably, the stimulus is not switched on abruptly but is slowly increased before each sequence 86 or advertisement break and decreased slowly after the end of each sequence 86 or advertisement break. Typically, the stimulus is increased linearly over a 30-60 second epoch prior to the image block or advertisement break so that it reaches its maximum value 60 seconds prior to the first image sequence or advertisement. Immediately the sequence of reference images of the reference period 95 ends, the stimulus intensity is linearly reduced to the minimum value over a 30 second period. The slow linear increase and decrease of stimulus intensity occurs for every sequence 86 or advertisement break.

### General Brand Characteristics

Once SSVEP or SSVER phase increase has been recorded from all subjects, the activity associated with each specific image sequence is averaged across trials for each subject and then across all subjects. Preferably, image sequences presented before and after the advertisement breaks are averaged separately. The General Brand Characteristics or the psychological response to the brand alone is indicated by the peak value of the SSVEP or SSVER phase increase at the above listed scalp sites during the period that the brand or product image is presented alone. More specifically, peak SSVEP or SSVER phase increase is assessed during brand image period 92 of FIG. 24, from which is subtracted SSVEP or SSVER phase increase assessed during the reference period 95. The psychological responses to the brand or product image thus include: the level of Attention to Detail elicited by the brand or product image; the level of Attention to Global Features elicited by the brand or product image; the level of Desirability elicited by the brand or product image; the level of Emotional Intensity elicited by the brand or product image; the level of Memory Encoding for Text and Detail elicited by the brand or product image; the level of Memory Encoding for Emotions or Imagery elicited by the brand or product image; the extent to which the brand or product image elicits feelings of Attraction or Repulsion; and the extent to which the brand or product image Engages subjects. These responses are determined as described herein.

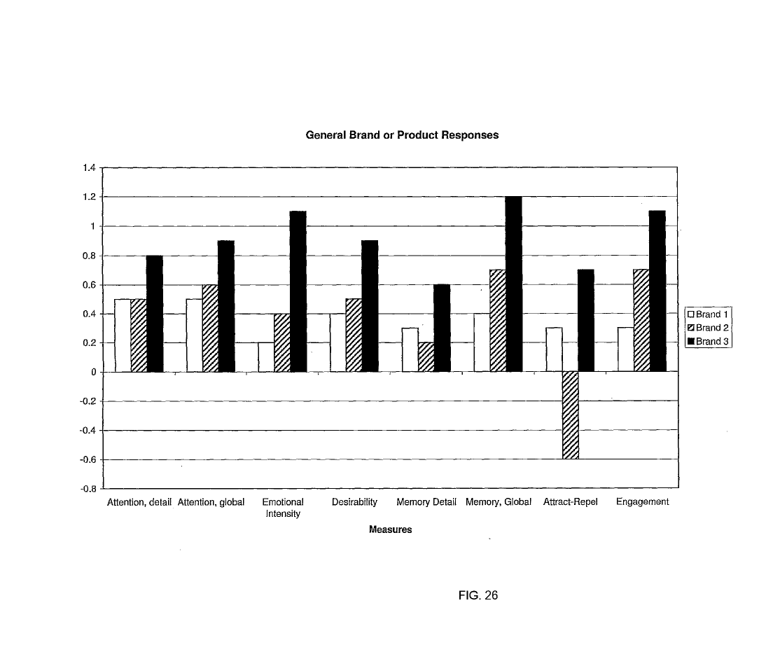

FIG. 26 illustrates the peak value of the above measures for three hypothetical brands, Brand 1, Brand 2 and Brand 3. In this example, Brand 1 is a frozen vegetable product brand, Brand 2 a tobacco product brand, while Brand 3 is a global airline and mobile phone brand. While Brand 1 elicits low to moderate levels of the various measures (as labeled in FIG. 26), Brand 2 elicits a higher level of Emotional Intensity, Global Memory Encoding and Engagement and a strong Repulsion. Brand 3 elicits the strongest levels of Attention, Emotional Intensity, Emotional Memory, Engagement and a strong Attraction. This data would inform corporate brand managers that Brand 1 has a relatively weak brand presence that is generally neutral to positive. Brand 2, on the other hand elicits stronger Emotional Intensity and Engagement indicating a strong emotional presence. However, subjects are Repelled by the brand indicating an active dislike of the brand. By contrast, Brand 3 elicits very high levels of Attention, Emotional Intensity, Engagement and strong Attraction. This brand has a very high presence that is very positive. These data would indicate that the subject group considers Brand 1 of little personal relevance and a weak motivator for brand loyalty. Brand 2 is negatively perceived and the subject group would actively avoid this brand. Brand 3 has a very strong and positive brand presence that is consistent with subjects having feelings of high brand loyalty to Brand 3.

The General Brand Characteristics can be measured a number of times to examine the change in these Brand Characteristics. The impact of an advertisement or the television program can be assessed by determining the change in Brand Characteristics or Brand Characteristics after viewing television program or advertisement or program minus Brand Characteristics before viewing television program or advertisement.

Long term changes in brand perception can also be assessed by measuring Brand Characteristics repeatedly over a period of time. These are termed Brand Characteristic tracking studies and the period between measurements can vary from weeks (for advertisement tracking) to months (for brand tracking).

### Example 8

### Specific Brand Characteristics

The congruence between ideas and feelings associated with a brand and a specific quality, or Specific Brand Characteristics can be determined from the brain responses elicited by the simultaneous appearance of the brand or product image and the semantic probe. Specific brand characteristics can be determined by reference to differences between the reference level of activity during the reference period 95 and SSVEP or SSVER phase increase when viewing the Image-semantic probe combinations during the congruence periods 94. In this Example, 50 subjects viewed twenty corporate logos (representing brands) in the periods 92 and each logo was presented twice followed by congruence periods 94 in which one of the congruence periods included a semantic probe which was generally consistent with the perception of the brand followed by congruence periods in which the semantic probe was generally inconsistent with the perception of the brand. Responses to congruent and incongruent combinations were averaged separately across trials and individuals. While congruent combinations elicited an increase or positive measure of activity at this site, incongruent combinations gave rise to a reduction or a negative measure of activity.

The congruence between the brand or product image and the semantic probe is indicated by the peak value of SSVEP or SSVER phase increase at the right prefrontal site located in the vicinity of electrode Fₚ₂ in the International 10-20 system. If inverse mapping techniques are used, the relevant cortical location is the right orbitofrontal cortex in the vicinity of Brodmann area 11.

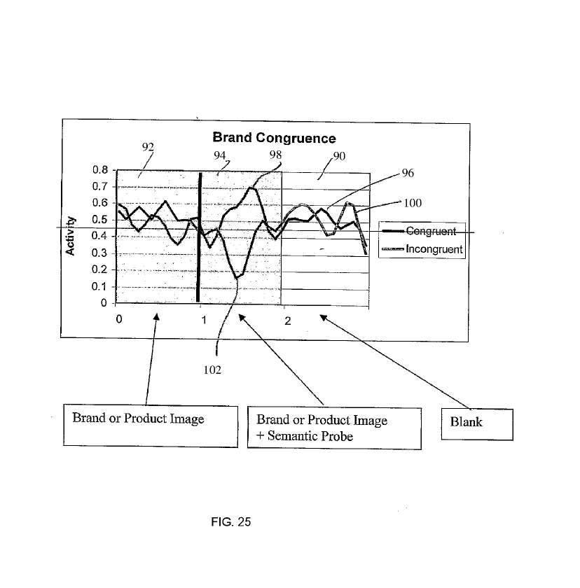

FIG. 25 illustrates brand congruence as determined for one of the twenty corporate logos which were included in the sequence 86. Similar graphical results could be produced for the other nineteen corporate logos but it is unnecessary to describe all of these in detail. More particularly, FIG. 25 shows the period 92 in which the brand or product image is shown followed by the congruence period 94 in which the brand or product image and semantic probe are shown followed by the blank period 90. In this case each of the periods 92, 84 and 90 is of 1 second duration. The line 96 indicates congruence between the semantic probe and the subjects' perception of the brand or product image. It will be seen that the line 96 includes a peak 98 which indicates strong consistency between the semantic probe and the subject's perception of the brand or product image.

FIG. 25 also shows a line 100 which illustrates incongruence between the brand or product image and the semantic probe. For instance, if the product were an automobile noted for safety, the semantic probe could be the word “unsafe” and this generates a trough 102 indicating incongruence between the subjects' perception of the brand or product image and the semantic probe. The ability to measure incongruence is a useful tool for clients to assess the perception of brands or product images against various adverse characteristics, as indicated by the semantic probe.

### Example 9

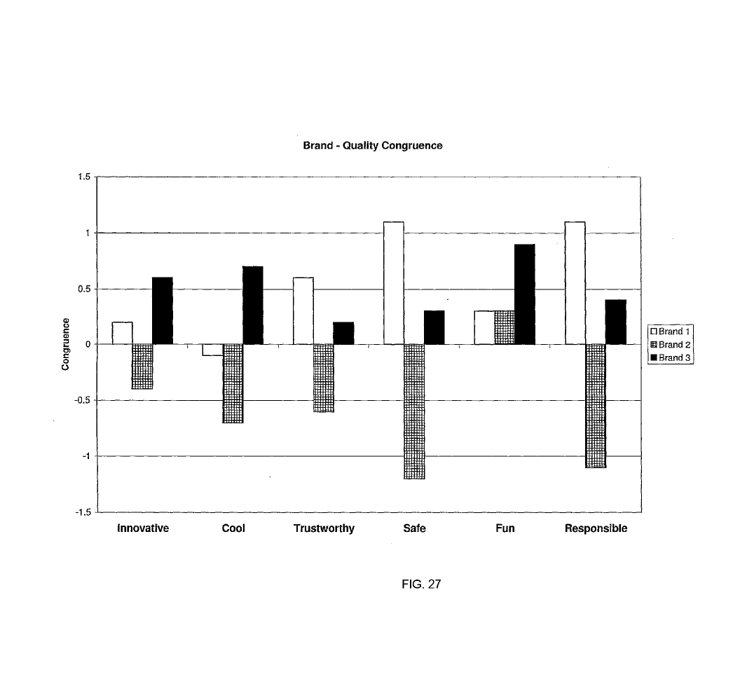

This Example is similar to Example 8 except that three hypothetical brands were included in the brand periods 90 and six different semantic probes were included in the congruence periods 94. SSVEP or SSVER phase increases were recorded during each of the periods 90, 92 and 94 as well as the reference periods 95. FIG. 27 illustrates graphically the congruence measure between the six semantic probes, “Innovative,” “Cool,” “Trustworthy,” “Safe,” “Fun,” and “Responsible” and the three hypothetical brands (Brand 1, a vehicle brand known for its emphasis on safety, Brand 2 a cigarette brand, and Brand 3, an airline and mobile phone brand. The graphical results indicate that Brand 1 is viewed by the subjects as trustworthy, safe and responsible, while lacking in fashion or fun as indicated by negative or low levels to the semantic probes “cool” and “fun.” Brand 2 is viewed very negatively as unfashionable, unsafe and untrustworthy as indicated by strongly negative assessments to all the semantic probes except the word “fun,” which is low positive. Brand 3 is viewed as fashionable and fun as indicated by high positive responses to the semantic probes “cool” and “fun.”

These measures offer brand managers an objective and more accurate indication of the way a brand is perceived and also changes in brand perception. Such changes may be a result of actions taken by the company such as advertising or sponsorship or may be a consequence of desired or undesired publicity. Any undesired changes in brand perception can be detected early and appropriate action taken.

Many modifications will be apparent to those skilled in the art without departing from the spirit and scope of the invention.

1-181. (canceled)

182. A method of quantitatively assessing at least one psychological response of at least one subject to at least one stimulus including the steps of:
(a) presenting the at least one stimulus to the at least one subject, the at least one stimulus having a sequence of audio, visual and/or audiovisual features that occur as a function of time;
(b) obtaining, during the presentation of the at least one stimulus, electroencephalogram (EEG) signals and/or magnetoencephalogram (MEG) signals from at least one pre-determined scalp site on the at least one subject;
(c) calculating steady state visually evoked potential (SSVEP) phase increase and/or steady state visually evoked response (SSVER) phase increase from said EEG and/or MEG signals to obtain output signals representing pre-determined psychological states of the at least one subject occurring as a function of time;
(d) optionally combining the output signals from a plurality of subjects to obtain pooled output signals; and
(e) analyzing the output signals and/or pooled output signals to quantitatively assess the at least one subject's at least one psychological response to the at least one stimuli.

183. The method of claim 182 wherein the at least one stimulus is selected from the group consisting of entertainment material, commercial communication, at least one character and combinations thereof.

184. The method of claim 182, further including the step of presenting a semantic probe simultaneously with step 182 (a).

185. The method of claim 182, further including the following steps: presenting at least one stimulus different from the at least one stimulus in (a) to the at least one subject; and waiting for a pre-determined period of time.

186. The method of claim 184 wherein reference stimulus is presented to the at least one subject.

187. The method of claim 186 further including the steps of claim 182 (b)-(e), except that the EEG and/or MEG signals obtained, and the quantitative assessment analyzed is for the reference stimulus.

188. The method of claim 182 wherein the at least one stimulus has a set of pre-determined parameters.

189. The method of claim 187, further including the steps of tracking the gaze position of at least one eye of the at least one subject during the claim 182 (a) step; and determining the difference in SSVEP and/or SSVER phase from step 182 (c) and the SSVEP and/or SSVER phase calculated for the reference stimulus according to claim 187.

190. The method of claim 182 wherein said at least one pre-determined scalp site is selected to quantitatively assess at least one psychological state selected from the group consisting of visual attention to detail, visual attention to global features, multi-modal attention to detail, desirability, multi-modal attention to global features, emotional intensity, long-term memory encoding, attraction-repulsion, engagement, likeability, behavioral intent, and combinations thereof.

191. The method of claim 182 including the steps of applying a sinusoidally varying flicker stimulus to the at least one subject during presentation of the at least one stimulus, and calculating Fourier coefficients from said output signals.

192. The method of claim 191 wherein the flicker stimulus is applied only to the peripheral vision of the at least one subject.

193. The method of claim 182 wherein application of said flicker stimulus comprises peripherally directing light through bilateral screens toward the eyes of each at least one subject, said screens each including an opaque area, and wherein said screens are positioned relative to each subject such that said opaque areas prevent said flicker stimulus from impinging on the fovea of each eye of each subject.

194. The method of claim 193 wherein the opacity of each screen decreases as a function of distance from its opaque area so that the intensity of the flicker stimulus impinging on each retina of each subject decreases in value from central vision to peripheral vision.

195. The method of claim 194 including the step of applying a masking pattern to each screen to define the opacity thereof, wherein the pattern is applied in accordance with a masking pattern function which provides zero or low gradients for changes in opacity adjacent to its opaque area and peripheral areas thereof.

196. The method of claim 195 wherein the opaque area of each screen is circular and wherein the masking pattern function is selected to be a Gaussian function, so that the opacity P of the screen is defined by the equation:
$$P = {e}^{- {( r - R )}^{2} / {G}^{2}}$$
where:
r is the radial distance from the centre of the opaque area; and
G is a parameter that determines the rate of fall-off of opacity with radial distance, and wherein when r<R, P=1.

197. The method of claim 196 wherein G has a value in the range R/4 and 2R.

198. The method of claim 182 wherein SSVEP phase and/or SSVER phase is calculated by the equations:
(a) calculation of SSVEP and/or SSVER amplitude and phase coefficients for each stimulus cycle for a given stimulus frequency using Fourier technique:
$${a}_{n} = \frac{1}{S Δ τ} {\sum }_{i = 0}^{S - 1} f ( nT + i Δ τ ) cos ( \frac{2 π}{T} ( nT + i Δ τ ) )$$
$${b}_{n} = \frac{1}{S Δ τ} {\sum }_{i = 0}^{S - 1} f ( nT + i Δ τ ) sin ( \frac{2 π}{T} ( nT + i Δ τ ) )$$
(b) calculation of SSVEP And/or SSVER Fourier components where aₙ and bₙ are the cosine and sine Fourier coefficients respectively, n represents the nth stimulus cycle, S is the number of samples per stimulus cycle, Δτ is the time interval between samples, T is the period of one cycle and f(nT+iΔτ) is the EEG or MEG signal:
$${SSVEP}_{amplitude} = \sqrt{( {A}_{n}^{2} + {B}_{n}^{2} )} or {SSVER}_{amplitude} = \sqrt{( {A}_{n}^{2} + {B}_{n}^{2} )}$$
$${SSVEP}_{phase} = atan ( \frac{{B}_{n}}{{A}_{n}} ) or {SSVER}_{phase} = atan ( \frac{{B}_{n}}{{A}_{n}} )$$
where N is the number of Fourier coefficients averaged, and where Aₙ and Bₙ are overlapping smoothed Fourier coefficients calculated by using:
(c)
$${A}_{n} = {\sum }_{i = 1}^{i = N} {a}_{n + i} / N$$
$${B}_{n} = {\sum }_{i = 1}^{i = N} {b}_{n + i} / N$$

199. A system for quantitatively assessing at least one psychological response of at least one subject to at least one stimulus including:
means for presenting the at least one stimulus to at least one subject, the at least one stimulus having a sequence of audio, visual and/or audiovisual features that occur as a function of time;
means for obtaining, during the presentation of the at least one stimulus, electroencephalogram (EEG) signals and/or magnetoencephalogram (MEG) signals from at least one pre-determined scalp site on the at least one subject; and
means for calculating steady state visually evoked potential (SSVEP) phase increase and/or steady state visually evoked response (SSVER) phase increase from said EEG and/or MEG signals to obtain output signals representing pre-determined psychological states of the at least one subject occurring as a function of time.

200. The system of claim 199 further comprising: a gaze tracking means for determining the gaze position of the at least one subject on said display means; and a detecting means for detecting when the gaze position of the at least one subject impinges on pre-determined visual features of the at least one stimulus.

201. The system of claim 199 further comprising a means for presenting a semantic probe.

202. A method of improving at least one psychological response of at least one subject to at least one stimulus including the steps of:
presenting an early version of the at least one stimulus to at least one subject;
quantitatively assessing at least one psychological response of the at least one subject in accordance with the method as claimed in claim 182; and
editing the at least one stimulus to modify features that are assessed to be unsatisfactory.
